SEQUENCE LISTING (27 sequences)

SEQ ID NO: 1 - protein, 295 aa, Homo sapiens
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 2 - protein, 295 aa, Mus musculus
MPRGPASLLLLVLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKTKNEDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 3 - protein, 295 aa, Rattus norvegicus
MLRGPASLLLLVLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPPTEEAPKVCEACKTKNEDDNDIMETLCKNDFALKIKVKEITYINRDTKIILETKSKTIYKLNGVSDRDLKKSVLWLKDSLQCTCEEMNDINAPYLVMGQKQGGELVITSVKRWQKGQREFKRISRSIRKLQC

SEQ ID NO: 4 - protein, 294 aa, Canis lupus
MPRGPGSLLLLVLASHCCLGSARGLFFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNEDDNDIMETLCKNDFALKIKVKEITYINRDTKIILETKSKTIYKLNGVSERDLKKSVLWLKDSLQCTCEEMNDINAPYLVMGQKLGGELVITSVKRWQKGQREFKRISRSIRKLQC

SEQ ID NO: 5 - protein, 294 aa, Bos taurus
MLQGPGSLLLIVLASHCCLGSARGLFFGQSDFPYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLDCDRFPQDNDLCIPLASSDHLLPATEEAPKVCEACKTKNEDDNDIMETLCKNDFALKIKVKEITYINRDTKIILETKSKTIYKLNGVSEKDLKKSVLWLKDSLQCTCEEMNDINAPYLVMGQKLGGELVITSVKRWQKGQREFKRISRSFRKLQC

SEQ ID NO: 6 - protein, 292 aa, Gallus gallus
MPRRLCALLLLASQCLGSTAGLFPFGEPDFSYKRSNCKPIPAPMLLCRGIEYQSMRLPNLLGHETVQEVLEQATTWIPLVQKQCHPDTRKFLCSLFAPVCIDDLDEIIQPCHSLCEEVKESCAPVMSAFGFPWPDMLDCSRFPKDNDLCIPLASSDHILPVTREAPKVCDACKNKNEDDNDIMENLCKNDFALKIKVKEIAYINGDTKITPETKSKTIYKLNGLTERDLRKIVLWLKGGLQCTCDEMNDINVPYLVMGQKQAGELVITSLKRWQKGQRAFKRFSRSIRKLQC

SEQ ID NO: 7 - protein, 9 aa, Artificial Sequence
  Synthetic amino acid sequence
YPYDVPDYA

SEQ ID NO: 8 - protein, 8 aa, Artificial Sequence
  Synthetic amino acid sequence
DYKDDDDK

SEQ ID NO: 9 - protein, 10 aa, Artificial Sequence
  Synthetic amino acid sequence
EQKLISEEDL

SEQ ID NO: 10 - protein, 5 aa, Artificial Sequence
  Synthetic amino acid sequence
HHHHH

SEQ ID NO: 11 - protein, 6 aa, Artificial Sequence
  Synthetic amino acid sequence
HHHHHH

SEQ ID NO: 12 - protein, 8 aa, Artificial Sequence
  Synthetic amino acid sequence
WSHPQFEK

SEQ ID NO: 13 - protein, 5 aa, Artificial Sequence
  Synthetic amino acid sequence
RYIRS

SEQ ID NO: 14 - protein, 4 aa, Artificial Sequence
  Synthetic amino acid sequence
FHHT

SEQ ID NO: 15 - protein, 17 aa, Artificial Sequence
  Synthetic amino acid sequence
WEAAAREACCRECCARA

SEQ ID NO: 16 - protein, 5 aa, Artificial Sequence
  Synthetic amino acid sequence
GSGGS

SEQ ID NO: 17 - protein, 5 aa, Artificial Sequence
  Synthetic amino acid sequence
GGGSN

SEQ ID NO: 18 - protein, 4 aa, Artificial Sequence
  Synthetic amino acid sequence
GGSG

SEQ ID NO: 19 - protein, 5 aa, Artificial Sequence
  Synthetic amino acid sequence
GGSGG

SEQ ID NO: 20 - protein, 5 aa, Artificial Sequence
  Synthetic amino acid sequence
GSGSG

SEQ ID NO: 21 - protein, 5 aa, Artificial Sequence
  Synthetic amino acid sequence
GSGGG

SEQ ID NO: 22 - protein, 5 aa, Artificial Sequence
  Synthetic amino acid sequence
GGGSG

SEQ ID NO: 23 - protein, 5 aa, Artificial Sequence
  Synthetic amino acid sequence
GSSSG

SEQ ID NO: 24 - DNA, 888 bp, murine
atgccgcggggccctgcctcgctgctgctgctagtcctcgcctcgcactgctgcctgggctcggcgcgtgggctcttcctcttcggccagcccgacttctcctacaagcgcagcaactgcaagcccatccccgccaacctgcagctgtgccacggcatcgagtaccagaacatgcggctgcccaacctgctgggccacgagaccatgaaggaggtgctggagcaggcgggcgcctggattccgctggtcatgaagcagtgccacccggacaccaagaagttcctgtgctcgctcttcgcccctgtctgtctcgacgacctagatgagaccatccagccgtgtcactcgctctgcgtgcaggtgaaggaccgctgcgccccggtcatgtccgccttcggcttcccctggccagacatgctggagtgcgaccgtttcccgcaggacaacgacctctgcatccccctcgctagtagcgaccacctcctgccggccacagaggaagctcccaaggtgtgtgaagcctgcaaaaccaagaatgaggacgacaacgacatcatggaaaccctttgtaaaaatgacttcgcactgaaaatcaaagtgaaggagataacgtacatcaacagagacaccaagatcatcctggagacaaagagcaagaccatttacaagctgaacggcgtgtccgaaagggacctgaagaaatccgtgctgtggctcaaagacagcctgcagtgcacctgtgaggagatgaacgacatcaacgctccgtatctggtcatgggacagaagcagggcggcgagctggtgatcacctccgtgaaacggtggcagaagggccagagagagttcaagcgcatctcccgcagcatccgcaagctgcaatgctag

SEQ ID NO: 25 - protein, 295 aa, murine; identical to SEQ ID NO: 2

SEQ ID NO: 26 - DNA, 20 bp, Artificial Sequence
  Primer
atgccgcggggccctgcctc

SEQ ID NO: 27 - DNA, 21 bp, Artificial Sequence
  Primer
ctagcattgcagcttgcggat

(19) United States
(12) Patent Application Publication
(10) Pub. No.: US 2013/0216546 A1
(43) Pub. Date: Aug. 22, 2013

(54) Methods of Treating Glucose Metabolism Disorders

(71) Applicant: NGM Biopharmaceuticals, Inc., (US)

(72) Inventors: Zhaodan Cao, San Antonio, TX (US); Yarong Lu, Watertown, MA (US)

(73) Assignee: NGM BIOPHARMACEUTICALS, INC., South San Francisco, CA (US)

(21) Appl. No.: 13/715,312

(22) Filed: Dec. 14, 2012

Related U.S. Application Data
(60) Provisional application No. 61/585,139, filed on Jan. 10, 2012.
(60) Provisional application No. 61/590,637, filed on Jan. 25, 2012.

(51) Int. Cl. A61K 39/395; A61K 45/06; C07K 16/18

(52) U.S. Cl. CPC A61K 39/3955; C07K 16/18; A61K 45/06
USPC 424/139.1; 530/387.9; 530/387.3; 530/391.3; 530/391.1

(57) ABSTRACT

Methods of treating individuals with a glucose metabolism disorder, and compositions suitable for use in the methods, are provided.

### CROSS-REFERENCE TO RELATED APPLICATIONS

This application claims priority benefit of U.S. provisional application Ser. No. 61/585,139, filed Jan. 10, 2012 and U.S. provisional application Ser. No. 61/590,637, filed Jan. 25, 2012, each of which applications is incorporated by reference herein in its entirety.

### INTRODUCTION

High blood glucose levels stimulate the secretion of insulin by pancreatic beta-cells. Insulin in turn stimulates the entry of glucose into muscles and adipose cells, leading to the storage of glycogen and triglycerides and to the synthesis of proteins. Activation of insulin receptors on various cell types diminishes circulating glucose levels by increasing glucose uptake and utilization, and by reducing hepatic glucose output. Disruptions within this regulatory network can result in diabetes and associated pathologic syndromes that affect a large and growing percentage of the human population.

Patients who have a glucose metabolism disorder can suffer from hyperglycemia, hyperinsulinemia, and/or glucose intolerance. An example of a disorder that is often associated with the aberrant levels of glucose and/or insulin is insulin resistance, in which liver, fat, and muscle cells lose their ability to respond to normal blood insulin levels.

Therapy that can modulate glucose and/or insulin levels in a patient and to enhance the biological response to fluctuating glucose levels remain of interest.

### SUMMARY

The present disclosure provides compositions and methods that find use in modulating glucose and/or insulin levels in glucose metabolism disorders. The present methods involve using an antibody targeting SFRP2 protein for modulating glucose metabolism. The agent may be used as therapy to treat various glucose metabolism disorders, such as diabetes mellitus, and/or obesity. The proteins targeted by the method of the present disclosure encompass those expressed by SFRP2 genes, and homologues thereof, and are useful for but not limited to treating one or more of the following conditions: diabetes mellitus (e.g. diabetes type I, diabetes type II and gestational diabetes), insulin resistance, hyperinsulinemia, glucose intolerance, hyperglycemia or metabolic syndrome.

The present disclosure provides a method of treating a subject, the method comprising: administering to the subject having a glucose metabolism disorder a therapeutically effective amount of an antibody that specifically binds to a protein comprising an amino acid sequence having at least 79% amino acid sequence identity (e.g., at least 79%, at least 85%, at least 90%, at least 95%, at least 97%, at least 98%, at least 99%, or 100%, amino acid sequence identity) to an amino acid sequence of human SFRP2, wherein the administering is effective to treat a symptom of a glucose metabolism disorder. In some cases, the glucose metabolism disorder comprises hyperglycemia and wherein the administering reduces plasma glucose in the subject. In some cases, the glucose metabolism disorder comprises hyperinsulinemia and wherein the administering reduces plasma insulin in said subject. In some cases, glucose metabolism disorder comprises glucose intolerance and wherein the administering increases glucose tolerance in the subject. In some cases, the glucose metabolism disorder comprises diabetes mellitus. In some cases, the subject is obese. In some cases, the glucose metabolism disorder is diet-induced. In any of these embodiments, the subject can be a human.

In carrying out the method, the administering is in some cases by parenteral injection. In some cases, the parenteral injection is subcutaneous.

The present disclosure provides a monoclonal antibody that binds specifically to an SFRP2 polypeptide comprising an amino acid sequence having at least 79% amino acid sequence identity (e.g., at least 79%, at least 85%, at least 90%, at least 95%, at least 97%, at least 98%, at least 99%, or 100%, amino acid sequence identity) to an amino acid sequence of human SFRP2. In some cases, the antibody comprises a light chain variable region and a heavy chain variable region present in separate polypeptides. In some cases, the antibody comprises a light chain variable region and a heavy chain variable region present in a single polypeptide. In some cases, the antibody binds the SFRP2 polypeptide with an affinity of from about 10⁷ M⁻¹ to about 10¹² M. In some cases, the antibody comprises heavy chain constant region, and wherein the heavy chain constant region is of the isotype IgG1, IgG2, IgG3, or IgG4. The antibody can be detectably labeled. The antibody can be a Fv, scFv, Fab, F(ab′)2, or Fab′.

In some cases, the antibody comprises a covalently linked non-peptide synthetic polymer. In some cases, the synthetic polymer is poly(ethylene glycol) polymer.

In some embodiments, a subject antibody comprises a covalently linked moiety selected from a lipid moiety, a fatty acid moiety, a polysaccharide moiety, and a carbohydrate moiety. In some cases, a subject antibody comprises an affinity domain. In some cases, a subject antibody is immobilized on a solid support. In some cases, a subject antibody is a humanized antibody. In some cases, a subject antibody is a single chain Fv (scFv) antibody. The scFv is in some instances multimerized.

The present disclosure provides a pharmaceutical composition that comprises a subject antibody; and a pharmaceutically acceptable excipient, diluent, or carrier. In some cases, the excipient is an isotonic injection solution. In some cases, the composition is suitable for human administration. The present disclosure provides a sterile container comprising a subject pharmaceutical composition. For example, the container can be a syringe. The present disclosure provides a kit comprising a subject sterile container. In some cases, the kit further comprises a second sterile container comprising a second therapeutic agent.

### BRIEF DESCRIPTION OF THE DRAWINGS

FIG. 1 shows body weight of mice on a high fat diet that were injected with an adeno-associated virus (AAV) expressing a protein of the present disclosure compared to those of mice injected with a control virus and those on a lean diet.

FIG. 2 shows blood glucose of mice on high fat diet that were injected with AAV expressing a protein of the present disclosure compared to those of mice injected with a control virus and those on a lean diet.

FIG. 3 shows insulin levels of mice on high fat diet that were injected with AAV expressing a protein of the present disclosure compared to those of mice injected with a control virus and those on a lean diet.

FIG. 4 shows the level of glucose in mice over 120 minute period post injection of 1 g/kg of glucose. Glucose tolerance was monitored in mice on a high fat diet that have been injected with AVV expressing a protein provided by the present disclosure or the control and in mice that were on a lean diet.

FIG. 5 shows the result of an insulin tolerance test. Glucose levels were monitored after an intraperitoneal injection of insulin (0.75 units/kg). Response to insulin was compared among DIO mice injected with AAV expressing a protein of the present disclosure and those injected with AAV expressing the control, as well as lean mice.

FIG. 6 shows an alignment of SFRP2 amino acid sequences (SEQ ID NOS:1-6) from humans, rodents, dogs, cow, and chickens.

FIG. 7 shows the level of blood glucose in mice injected with mAb106, a monoclonal antibody to human SFRP2.

FIG. 8 shows the level of plasma insulin in mice injected with mAb106, a monoclonal antibody to human SFRP2.

FIG. 9 shows the body weight of mice injected with mAb106, a monoclonal antibody to human SFRP2.

### DETAILED DESCRIPTION

Overview

The present disclosure provides, inter alia, antibodies and compositions thereof useful in modulating glucose and/or insulin levels in glucose metabolism disorders. The proteins targeted by the methods and compositions of the present disclosure encompass SFRP2 (secreted frizzled-related protein 2), also known as secreted apoptosis-related protein-1 (SARP1), FKSG12, unq361, or pro697, genes and/or proteins encoded thereby, and are useful for, e.g., conditions of glucose metabolism dysregulation such as, but not limited to treating diabetes mellitus (e.g. diabetes type I and diabetes type II). In a diet-induced obesity model (mice on a high fat diet), the glucose and insulin levels are higher than those in a subject on a regular lean diet. However, when the proteins of the present disclosure are administered (as exemplified by expression from an AAV), certain symptoms of glucose metabolism disorder are exacerbated. Accordingly, targeting the proteins of the present disclosure, either to decrease the amount and/or the activity of the mature protein may be used in restoring glucose homeostasis in subjects with a dysfunctional glucose metabolism, including subjects who may be overweight, obese, and/or on a high fat diet.

### DEFINITIONS

The terms “patient” or “subject” as used interchangeably herein in the context of therapy, refer to a human and non-human animal, as the recipient of a therapy or preventive care.

The phrase “in a sufficient amount to effect a change in” means that there is a detectable difference between a level of an indicator measured before and after administration of a particular therapy. Indicators include but are not limited to glucose and insulin.

The phrase “glucose tolerance”, as used herein, refers to the ability of a subject to control the level of plasma glucose and/or plasma insulin when glucose intake fluctuates. For example, glucose tolerance encompasses the ability to reduce, within about 120 minutes or so, the level of plasma glucose back to a level before the intake of glucose.

The phrase “pre-diabetes”, as used herein, refers to a condition that may be determined using, e.g., either the fasting plasma glucose test (FPG) or the oral glucose tolerance test (OGTT). Both require a subject to fast overnight (e.g., for at least 8 hours prior to initiating the test). In the FPG test, a person's blood glucose is measured after the conclusion of the fasting morning; generally the subject fasts overnight and the blood glucose is measured in the morning before the subject eats. In the OGTT, a subject's blood glucose is checked after fasting and again 2 hours after drinking a glucose-rich drink. In a healthy individual, a normal test result of FPG would indicate a glucose level of below about 100 mg/dl. A subject with pre-diabetes would have a FPG level between about 100 and about 125 mg/dl. If the blood glucose level rises to about 126 mg/dl or above, the subject is determined to have “diabetes”. In the OGTT, the subject's blood glucose is measured after a fast and 2 hours after drinking a glucose-rich beverage. Normal blood glucose in a healthy individual is below about 140 mg/dl 2 hours after the drink. In a pre-diabetic subject, the 2-hour blood glucose is about 140 to about 199 mg/dl. If the 2-hour blood glucose rises to 200 mg/dl or above, the subject is determined to have “diabetes”.

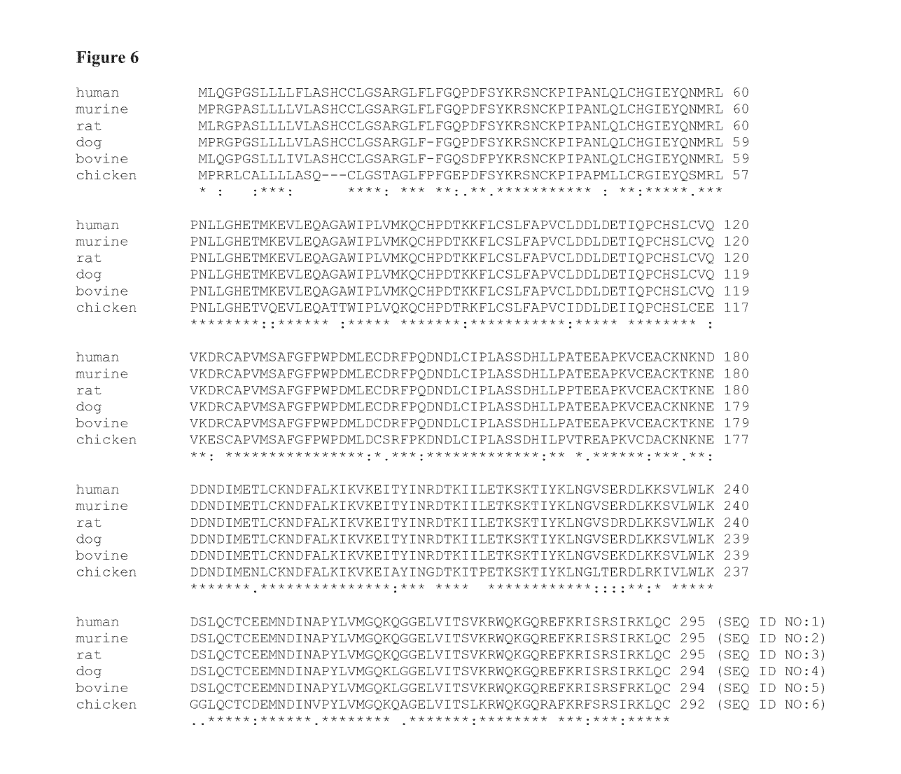

“SFRP2” (secreted frizzled-related protein 2), also known as SARP1, FKSG12, unq361, or PRO697, encompasses murine and human proteins that are encoded by gene SFRP2 or a gene homologue of SFRP2. SFRP2 is found in many mammals (e.g. human, non-human primates, canines, and mouse). See FIG. 6 for alignments of various amino acid sequences of SFRP2.

As used herein, “homologues” or “variants” refers to amino acid or DNA sequences that are similar to reference amino acid or nucleic acid sequences, respectively. Homologues or variants encompass naturally occurring DNA sequences and proteins encoded thereby and their isoforms. The homologues also include known allelic or splice variants of a protein/gene. Homologues and variants also encompass nucleic acid sequences that vary in one or more bases from a naturally-occurring DNA sequence but still translate into an amino acid sequence that corresponds to the naturally-occurring protein due to degeneracy of the genetic code. Homologues and variants may also refer to those that differ from the naturally-occurring sequences by one or more conservative substitutions and/or tags and/or conjugates.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

It will be appreciated that throughout this present disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

“The terms “nucleic acid,” “nucleic acid molecule,” “polynucleotide,” and the like are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers.

The term “heterologous” refers to two components that are defined by structures derived from different sources. For example, “heterologous” is used in the context of a polypeptide, where the polypeptide includes operably linked amino acid sequences that can be derived from different polypeptides (e.g., a first component consisting of a recombinant peptide and a second component derived from a native SFRP2 polypeptide). Similarly, “heterologous” in the context of a polynucleotide encoding a chimeric polypeptide includes operably linked nucleic acid sequence that can be derived from different genes (e.g., a first component from a nucleic acid encoding a peptide according to an embodiment disclosed herein and a second component from a nucleic acid encoding a carrier polypeptide). Other exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin relative to the promoter, the coding sequence or both). For example, a T7 promoter operably linked to a polynucleotide encoding a SFRP2 polypeptide or domain thereof is said to be a heterologous nucleic acid. “Heterologous” in the context of recombinant cells can refer to the presence of a nucleic acid (or gene product, such as a polypeptide) that is of a different genetic origin than the host cell in which it is present.

The term “operably linked” refers to functional linkage between molecules to provide a desired function. For example, “operably linked” in the context of nucleic acids refers to a functional linkage between nucleic acids to provide a desired function such as transcription, translation, and the like, e.g., a functional linkage between a nucleic acid expression control sequence (such as a promoter, signal sequence, or array of transcription factor binding sites) and a second polynucleotide, wherein the expression control sequence affects transcription and/or translation of the second polynucleotide. “Operably linked” in the context of a polypeptide refers to a functional linkage between amino acid sequences (e.g., of different domains) to provide for a described activity of the polypeptide.

As used herein in the context of the structure of a polypeptide, “N-terminus” and “C-terminus” refer to the extreme amino and carboxyl ends of the polypeptide, respectively, while “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from” in the context of an amino acid sequence or polynucleotide sequence (e.g., an amino acid sequence “derived from” a SFRP2 polypeptide) is meant to indicate that the polypeptide or nucleic acid has a sequence that is based on that of a reference polypeptide or nucleic acid (e.g., a naturally occurring SFRP2 polypeptide or SFRP2-encoding nucleic acid), and is not meant to be limiting as to the source or method in which the protein or nucleic acid is made. By way of example, the term “derived from” includes homologues or variants of reference amino acid or DNA sequences.

“Isolated” refers to a protein of interest (e.g., an SFRP2 polypeptide; an anti-SFRP2 antibody) that, if naturally occurring, is in an environment different from that in which it may naturally occur. “Isolated” is meant to include proteins that are within samples that are substantially enriched for the protein of interest and/or in which the protein of interest is partially or substantially purified. Where the protein is not naturally occurring, “isolated” indicates the protein has been separated from an environment in which it was made by either synthetic or recombinant means.

“Enriched” means that a sample is non-naturally manipulated (e.g., by an experimentalist or a clinician) so that a protein of interest (e.g., an SFRP2 polypeptide; an anti-SFRP2 antibody) is present in a greater concentration (e.g., at least a three-fold greater, at least 4-fold greater, at least 8-fold greater, at least 64-fold greater, or more) than the concentration of the protein in the starting sample, such as a biological sample (e.g., a sample in which the protein naturally occurs or in which it is present after administration), or in which the protein was made (e.g., as in a bacterial protein and the like).

“Substantially pure” indicates that an entity (e.g., an SFRP2 polypeptide; an anti-SFRP2 antibody) makes up greater than about 50% of the total content of the composition (e.g., total protein of the composition) and typically, greater than about 60% of the total protein content. More typically, a “substantially pure” refers to compositions in which at least 75%, at least 85%, at least 90% or more of the total composition is the entity of interest (e.g. 95%, 98%, 99%, greater than 99%), of the total protein. In some cases, the protein will make up greater than about 90%, e.g., greater than about 95% of the total protein in the composition.

The term “antibody” (also used interchangeably with “immunoglobulin”) encompasses polyclonal and monoclonal antibody preparations where the antibody may be of any class of interest (e.g., IgM, IgG, and subclasses thereof), as well as preparations including hybrid antibodies, altered antibodies, F(ab′)₂ fragments, F(ab) molecules, Fv fragments, single chain Fv fragments (scFv), single chain antibodies, single domain antibodies (V_{H}H), chimeric antibodies, humanized antibodies, and antigen-binding fragments thereof which exhibit immunological binding properties of the parent antibody molecule. Antibodies that have inhibitory functions for their targets are of particular interest. The antibodies described herein may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a support (e.g., a solid support), such as a polystyrene plate or bead, test strip, and the like.

The term “binding” refers to a direct association between two molecules, due to, for example, covalent, electrostatic, hydrophobic, and ionic and/or hydrogen-bond interactions, including interactions such as salt bridges and water bridges. A subject anti-SFRP2 antibody binds specifically to an epitope within an SFRP2 polypeptide, e.g., with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. A subject antibody binds to an epitope present on an SFRP2 polypeptide with an affinity of from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M, or greater than 10⁻¹² M. Non-specific binding would refer to binding with an affinity of less than about 10⁻⁷ M, e.g., binding with an affinity of 10⁻⁶ M, 10⁻⁵ M, 10⁻⁴ M, etc.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” comprise a variable region at the NH₂-terminus and a kappa or lambda constant region at the COOH-terminus. Full-length immunoglobulin “heavy chains” similarly comprise a variable region and one of the aforementioned heavy chain constant regions, e.g., gamma.

An immunoglobulin light or heavy chain variable region is composed of a “framework” region (FR) interrupted by three hypervariable regions, also called “complementarity determining regions” or “CDRs”. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996). Methods to define CDRs are available in the art and routinely performed. For example, framework regions and CDRs may be defined by IMGT (see, “Sequences of Proteins of Immunological Interest,” E. Kabat et al., U.S. Department of Health and Human Services, (1991 and Lefranc et al. IMGT, the international ImMunoGeneTics information System®. Nucl. Acids Res., 2005, 33, D593-D597)). A detailed discussion of the IMGTS system, including how the IMGTS system was formulated and how it compares to other systems, is provided on the World Wide Web at imgt.cines.fr/textes/IMGTScientificChart/Numbering/IMGTnumberingsTable.html. The framework region of an antibody, that is the combined framework regions of the constituent light and heavy chains, serves to position and align the CDRs. The CDRs are primarily responsible for binding to an epitope of an antigen.

The term “monoclonal antibody” refers to an antibody composition having a homogeneous antibody population. The term is not limited by the manner in which it is made. The term encompasses whole immunoglobulin molecules, as well as Fab molecules, F(ab′)2 fragments, Fv fragments, scFv, fusion proteins comprising an antigen-binding portion of an antibody and a non-antibody protein, and other molecules that exhibit immunological binding properties of the parent monoclonal antibody molecule. Methods of making polyclonal and monoclonal antibodies are known in the art.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CHO of the heavy chain. Fab fragments differ from Fab′ fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

Anti-SFRP2 Antibody

Methods and compositions are provided herein to, e.g., regulate glucose metabolism (e.g. glucose levels, insulin levels, glucose tolerance) in a subject. The subject methods and/or compositions target SFRP2 so as to decrease the amount and/or the activity of the mature SFRP2 protein.

The present disclosure provides methods for reducing (e.g., inhibiting, or neutralizing) SFRP2 activity in a subject with a glucose metabolism dysregulation. One way SFRP2 can be inhibited is to administer anti-SFRP2 antibodies (also referred to as “SFRP2 antibodies”), where such antibodies include a whole antibody (e.g. IgG), an antigen-binding fragment thereof, single-chain Fab, or a synthetic SFRP2 antibody that comprise portions of an antibody. SFRP2, the target of such antibodies, is implicated as a modulator in Wnt signaling. Details on SFRP2 are described below.

Administration of SFRP2 to mice with diet-induced obesity leads to an increase in serum insulin levels. Thus, SFRP2 can exacerbate symptoms of glucose metabolism (e.g. hyperinsulinemia). Accordingly, decreasing the amount and/or activity of SFRP2 can serve to treat glucose metabolism disorders.

Targeting SFRP2 as provided by the present methods can encompass inhibiting other biomolecules that normally interact with SFRP from binding to SFRP2 or sequestering SFRP2 from its signaling partners. For example, a subject anti-SFRP2 antibody can decrease SFRP2 activity, e.g., decrease SFRP2 interaction with the components of the WNT signaling pathway. The binding of a subject anti-SFRP2 antibody may be competitive or non-competitive with the endogenous binding partners of SFRP2. A subject anti-SFRP2 antibody may also modify the activity and/or structure of SFRP2. The methods can further encompass decreasing the level of SFRP2 expression and/or amount of mature, active SFRP. The method can also increase turnover of SFRP2.

A subject anti-SFRP2 antibody can reduce binding of an SFRP2 polypeptide to Wnt. For example, a subject antibody can reduce binding of an SFRP2 polypeptide to a Wnt polypeptide by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the degree of binding between the SFRP2 polypeptide and the Wnt polypeptide in the absence of the antibody.

In certain embodiments, an antibody comprising: a) a variable domain comprising: i. a CDR1 region that is identical in amino acid sequence to the heavy chain CDR1 region of an anti-SFRP2 antibody; ii. a CDR2 region that is identical in amino acid sequence to the heavy chain CDR2 region of the anti-SFRP2 antibody; and iii. a CDR3 region that is identical in amino acid sequence to the heavy chain CDR3 region of the anti-SFRP2 antibody; and b) a light chain variable domain comprising: i. a CDR1 region that is identical in amino acid sequence to the light chain CDR1 region of the anti-SFRP2 antibody; ii. a CDR2 region that is identical in amino acid sequence to the light chain CDR2 region of the anti-SFRP2 antibody; and iii. a CDR3 region that is identical in amino acid sequence to the light chain CDR3 region of the anti-SFRP2 antibody; or b) a variant of the variable domain of part a) that is otherwise identical to the variable domain of part a) except for a number of (e.g., 1, 2, 3, 4, 5, 6, 7 or 8) amino acid substitutions in the CDR regions, where the antibody binds an SFRP2 polypeptide.

In some embodiments, a subject antibody comprises: a) a light chain region comprising: i) one, two, or three complementarity determining regions (CDRs) from a mouse monoclonal anti-SFRP2 antibody light chain variable region sequence; and ii) a light chain framework region, e.g., a framework region from a human immunoglobulin light chain; and b) a heavy chain region comprising: i) one, two, or three CDRs from the mouse monoclonal anti-SFRP2 antibody heavy chain variable region sequence; and ii) a heavy chain framework region, e.g., a framework region from a human immunoglobulin heavy chain.

A subject anti-SFRP2 antibody can find use in a variety of applications, including use in various methods of treating a host suffering from a disease or condition associated with glucose metabolism, as well as in diagnosis of various diseases and conditions associated with SFRP2 expression.

Recombinant Antibody

A subject anti-SFRP2 antibody may be recombinant. The antibody may contain a light and/or heavy chain. Methods for producing recombinant antibodies are known in the art. For example, the nucleic acids encoding the antibody, or at least a complementary determining region (CDR) of a heavy chain polypeptide or at least a CDR of a light chain polypeptide, are introduced directly into a host cell, and the cell incubated under conditions sufficient to induce expression of the encoded antibody. The recombinant antibody may be glycosylated by an endogenous glycosylase in the host cells, unglycosylated, or may have an altered glycosylation pattern.

Where the antibody is recombinant, the antibody may be chimeric. Chimeric antibodies are immunoglobulin molecules comprising human and non-human portions. More specifically, the antigen combining region (or variable region) of a humanized chimeric antibody is derived from a non-human source (e.g. murine), and the constant region of the chimeric antibody (which confers biological effector function to the immunoglobulin) is derived from a human source. The chimeric antibody can have the antigen binding specificity of the non-human antibody molecule and the effector function conferred by the human antibody molecule. A large number of methods of generating chimeric antibodies are well known to those of skill in the art. An alternative approach is the generation of humanized antibodies by linking the CDR regions of non-human antibodies to human constant regions by recombinant DNA techniques.

A recombinant fusion antibody that is specific for a SFRP2 is contemplated, in which the antibody is modified to include a heterologous protein. For example, a heavy chain polypeptide and/or light chain polypeptide may be joined to a reporter protein or to a protein having a desired therapeutic effect. The reporter protein may be a fluorescent protein. The antibody may also be conjugated to a second antibody (or at least an antigen-binding portion thereof). Methods for producing a fusion protein of interest when provided a nucleic acid sequence are well known in the art.

Humanized and Human Antibodies

A subject anti-SFRP2 antibody will in some embodiments be humanized. Amino acids may be substituted in the framework regions of a parent non-human (e.g., mouse monoclonal) antibody to produce a modified antibody that is less immunogenic in a human than the parent non-human antibody. Antibodies can be humanized using a variety of techniques known in the art. Framework substitutions are identified by modeling of the interactions of the CDR and framework residues to identify framework residues important for antigen binding and sequence comparison to identify unusual framework residues at particular positions.

The antibody may also be a fully human antibody. Human antibodies are primarily composed of characteristically human polypeptide sequences. A subject human antibody can be produced by a wide variety of methods. For example, human antibodies can be produced initially in trioma cells (descended from three cells, two human and one mouse). Genes encoding the antibodies are then cloned and expressed in other cells, particularly non-human mammalian cells. The general approach for producing human antibodies by trioma technology has been described in the art.

Accordingly, the present disclosure contemplates a DNA molecule comprising a nucleic acid sequence encoding an antibody that binds to SFRP2. The disclosure further contemplates recombinant host cells containing an exogenous polynucleotide encoding at least a CDR of a heavy chain polypeptide or at least a CDR of a light chain polypeptide of the subject antibody.

scFv

In some embodiments, a subject antibody comprises anti-SFRP2 antibody heavy chain CDRs and anti-SFRP2 antibody light chain CDRs in a single polypeptide chain, e.g., in some embodiments, a subject antibody is a scFv. In some embodiments, a subject antibody comprises, in order from N-terminus to C-terminus: a first amino acid sequence of from about 5 amino acids to about 25 amino acids in length; a heavy chain CDR1 of an anti-SFRP2 antibody; a second amino acid sequence of from about 5 amino acids to about 25 amino acids in length; a heavy chain CDR2 of an anti-SFRP2 antibody; a third amino acid sequence of from about 5 amino acids to about 25 amino acids in length; a heavy chain CDR3 of an anti-SFRP2 antibody; a fourth amino acid sequence of from about 5 amino acids to about 25 amino acids in length; a light chain CDR1 of an anti-SFRP2 antibody; a fifth amino acid sequence of from about 5 amino acids to about 25 amino acids in length; a light chain CDR2 an anti-SFRP2 antibody; a sixth amino acid sequence of from about 5 amino acids to about 25 amino acids in length; a light chain CDR3 an anti-SFRP2 antibody; and a seventh amino acid sequence of from about 5 amino acids to about 25 amino acids in length.

In some embodiments, a subject anti-SFRP2 antibody comprises scFv multimers. For example, in some embodiments, a subject antibody is an scFv dimer (e.g., comprises two tandem scFv (scFv₂)), an scFv trimer (e.g., comprises three tandem scFv (scFv₃)), an scFv tetramer (e.g., comprises four tandem scFv (scFv₄)), or is a multimer of more than four scFv (e.g., in tandem). The scFv monomers can be linked in tandem via linkers of from about 2 amino acids to about 10 amino acids in length, e.g., 2 aa, 3 aa, 4 aa, 5 aa, 6 aa, 7 aa, 8 aa, 9 aa, or 10 aa in length. Suitable linkers include, e.g., (Gly)ₓ, where x is an integer from 2 to 10. Other suitable linkers are those discussed above. In some embodiments, each of the scFv monomers in a subject scFV multimer is humanized, as described above.

Antibody Modifications

A subject anti-SFRP2 antibody can comprises one or more modifications.

In some embodiments, a subject antibody comprises a free thiol (—SH) group at the carboxyl terminus, where the free thiol group can be used to attach the antibody to a second polypeptide (e.g., another antibody, including a subject antibody), a scaffold, a carrier, etc.

In some embodiments, a subject antibody comprises one or more non-naturally occurring amino acids. In some embodiments, the non-naturally encoded amino acid comprises a carbonyl group, an acetyl group, an aminooxy group, a hydrazine group, a hydrazide group, a semicarbazide group, an azide group, or an alkyne group. See, e.g., U.S. Pat. No. 7,632,924 for suitable non-naturally occurring amino acids. Inclusion of a non-naturally occurring amino acid can provide for linkage to a polymer, a second polypeptide, a scaffold, etc. For example, a subject antibody linked to a water-soluble polymer can be made by reacting a water-soluble polymer (e.g., PEG) that comprises a carbonyl group to a subject antibody that comprises a non-naturally encoded amino acid that comprises an aminooxy, hydrazine, hydrazide or semicarbazide group. As another example, a subject antibody linked to a water-soluble polymer can be made by reacting a subject antibody that comprises an alkyne-containing amino acid with a water-soluble polymer (e.g., PEG) that comprises an azide moiety; in some embodiments, the azide or alkyne group is linked to the PEG molecule through an amide linkage. A “non-naturally encoded amino acid” refers to an amino acid that is not one of the 20 common amino acids or pyrolysine or selenocysteine. Other terms that may be used synonymously with the term “non-naturally encoded amino acid” are “non-natural amino acid,” “unnatural amino acid,” “non-naturally-occurring amino acid,” and variously hyphenated and non-hyphenated versions thereof. The term “non-naturally encoded amino acid” also includes, but is not limited to, amino acids that occur by modification (e.g. post-translational modifications) of a naturally encoded amino acid (including but not limited to, the 20 common amino acids or pyrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

In some embodiments, a subject antibody is linked (e.g., covalently linked) to a polymer (e.g., a polymer other than a polypeptide). Suitable polymers include, e.g., biocompatible polymers, and water-soluble biocompatible polymers. Suitable polymers include synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g. a homo- or hetero-polysaccharide. Suitable polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

Suitable polymers can have an average molecular weight in a range of from 500 Da to 50000 Da, e.g., from 5000 Da to 40000 Da, or from 25000 to 40000 Da. For example, in some embodiments, where a subject antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

As noted above, in some embodiments, a subject antibody is covalently linked to a PEG polymer. In some embodiments, a subject scFv multimer is covalently linked to a PEG polymer. See, e.g., Albrecht et al. (2006) J. Immunol. Methods 310:100. Methods and reagents suitable for PEGylation of a protein are well known in the art and may be found in, e.g., U.S. Pat. No. 5,849,860. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

The PEG conjugated to the subject antibody can be linear. The PEG conjugated to the subject protein may also be branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

A subject antibody can be glycosylated, e.g., the antibody can comprise a covalently linked carbohydrate or polysaccharide moiety. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to an antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of an antibody.

A subject antibody will in some embodiments comprise a “radiopaque” label, e.g. a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives (see, e.g., U.S. Pat. No. 5,939,045), radiopaque multiurethanes (see U.S. Pat. No. 5,346,981), organobismuth composites (see, e.g., U.S. Pat. No. 5,256,334), radiopaque barium multimer complexes (see, e.g., U.S. Pat. No. 4,866,132), and the like.

A subject antibody can be covalently linked to a second moiety (e.g., a lipid, a polypeptide other than a subject antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

A subject antibody can be immobilized on a solid support. Suitable supports are well known in the art and comprise, inter alia, commercially available column materials, polystyrene beads, latex beads, magnetic beads, colloid metal particles, glass and/or silicon chips and surfaces, nitrocellulose strips, nylon membranes, sheets, duracytes, wells of reaction trays (e.g., multi-well plates), plastic tubes, etc. A solid support can comprise any of a variety of substances, including, e.g., glass, polystyrene, polyvinyl chloride, polypropylene, polyethylene, polycarbonate, dextran, nylon, amylose, natural and modified celluloses, polyacrylamides, agaroses, and magnetite. Suitable methods for immobilizing a subject antibody onto a solid support are well known and include, but are not limited to ionic, hydrophobic, covalent interactions and the like. Solid supports can be soluble or insoluble, e.g., in aqueous solution. In some embodiments, a suitable solid support is generally insoluble in an aqueous solution.

A subject antibody will in some embodiments comprise a detectable label. Suitable detectable labels include any composition detectable by spectroscopic, photochemical, biochemical, immunochemical, electrical, optical or chemical means. Suitable detectable labels include, but are not limited to, magnetic beads (e.g. Dynabeads™), fluorescent dyes (e.g., fluorescein isothiocyanate, texas red, rhodamine, a green fluorescent protein, a red fluorescent protein, a yellow fluorescent protein, and the like), radiolabels (e.g., ³H, ¹²⁵I, ³⁵S, ¹⁴C, or ³²P), enzymes (e.g., horse radish peroxidase, alkaline phosphatase, luciferase, and others commonly used in an enzyme-linked immunosorbent assay (ELISA)), and colorimetric labels such as colloidal gold or colored glass or plastic (e.g. polystyrene, polypropylene, latex, etc.) beads.

In some embodiments, a subject antibody comprises a contrast agent or a radioisotope, where the contrast agent or radioisotope is one that is suitable for use in imaging, e.g., imaging procedures carried out on humans. Non-limiting examples of labels include radioisotope such as ¹²³¹I (iodine), ¹⁸F (fluorine), ⁹⁹Tc (technetium), ¹¹¹In (indium), and ⁶⁷Ga (gallium), and contrast agent such as gadolinium (Gd), dysprosium, and iron. Radioactive Gd isotopes (¹⁵³Gd) also are available and suitable for imaging procedures in non-human mammals. A subject antibody can be labeled using standard techniques. For example, a subject antibody can be iodinated using chloramine T or 1,3,4,6-tetrachloro-3α,6α-dephenylglycouril. For fluorination, fluorine is added to a subject antibody during the synthesis by a fluoride ion displacement reaction. See, Muller-Gartner, H., TIB Tech., 16:122-130 (1998) and Saji, H., Crit. Rev. Ther. Drug Carrier Syst., 16(2):209-244 (1999) for a review of synthesis of proteins with such radioisotopes. A subject antibody can also be labeled with a contrast agent through standard techniques. For example, a subject antibody can be labeled with Gd by conjugating low molecular Gd chelates such as Gd diethylene triamine pentaacetic acid (GdDTPA) or Gd tetraazacyclododecanetetraacetic (GdDOTA) to the antibody. See, Caravan et al., Chem. Rev. 99:2293-2352 (1999) and Lauffer et al., J. Magn. Reson. Imaging, 3:11-16 (1985). A subject antibody can be labeled with Gd by, for example, conjugating polylysine-Gd chelates to the antibody. See, for example, Curtet et al., Invest. Radiol., 33(10):752-761 (1998). Alternatively, a subject antibody can be labeled with Gd by incubating paramagnetic polymerized liposomes that include Gd chelator lipid with avidin and biotinylated antibody. See, for example, Sipkins et al., Nature Med., 4:623-626 (1998).

Suitable fluorescent proteins that can be linked to a subject antibody include, but are not limited to, a green fluorescent protein from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304; e.g., Enhanced GFP, many such GFP which are available commercially, e.g., from Clontech, Inc.; a red fluorescent protein; a yellow fluorescent protein; any of a variety of fluorescent and colored proteins from Anthozoan species, as described in, e.g., Matz et al. (1999) Nature Biotechnol. 17:969-973; and the like.

A subject antibody will in some embodiments be linked to (e.g., covalently or non-covalently linked) a fusion partner, e.g., a ligand; an epitope tag; a peptide; a protein other than an antibody; and the like. Suitable fusion partners include peptides and polypeptides that confer enhanced stability in vivo (e.g., enhanced serum half-life); provide ease of purification, e.g., (His)ₙ, e.g., 6His, and the like; provide for secretion of the fusion protein from a cell; provide an epitope tag, e.g., GST, hemagglutinin (HA; e.g., YPYDVPDYA; SEQ ID NO:7), FLAG (e.g., DYKDDDDK; SEQ ID NO:8), c-myc (e.g., EQKLISEEDL; SEQ ID NO:9), and the like; provide a detectable signal, e.g., an enzyme that generates a detectable product (e.g., β-galactosidase, luciferase), or a protein that is itself detectable, e.g., a green fluorescent protein, a red fluorescent protein, a yellow fluorescent protein, etc.; provides for multimerization, e.g., a multimerization domain such as an Fc portion of an immunoglobulin; and the like.

The fusion may also include an affinity domain, including peptide sequences that can interact with a binding partner, e.g., such as one immobilized on a solid support, useful for identification or purification. Consecutive single amino acids, such as histidine, when fused to a protein, can be used for one-step purification of the fusion protein by high affinity binding to a resin column, such as nickel sepharose. Exemplary affinity domains include His5 (HHHHH) (SEQ ID NO:10), HisX6 (HHHHHH) (SEQ ID NO:11), C-myc (EQKLISEEDL) (SEQ ID NO:9), Flag (DYKDDDDK) (SEQ ID NO:8), StrepTag (WSHPQFEK) (SEQ ID NO:12), hemagglutinin, e.g., HA Tag (YPYDVPDYA; SEQ ID NO:7), glutathinone-5-transferase (GST), thioredoxin, cellulose binding domain, RYIRS (SEQ ID NO:13), Phe-His-His-Thr (SEQ ID NO:14), chitin binding domain, S-peptide, T7 peptide, SH2 domain, C-end RNA tag, WEAAAREACCRECCARA (SEQ ID NO:15), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, leucine zipper sequences, and maltose binding protein.

In some embodiments, a subject antibody is modified to include a carbohydrate moiety, where the carbohydrate moiety can be covalently linked to the antibody. In some embodiments, a subject antibody is modified to include a lipid moiety, where the lipid moiety can be covalently linked to the antibody. Suitable lipid moieties include, e.g., an N-fatty acyl group such as N-lauroyl, N-oleoyl, etc.; a fatty amine such as dodecyl amine, oleoyl amine, etc.; a C3-C16 long-chain aliphatic lipid; and the like. See, e.g., U.S. Pat. No. 6,638,513). In some embodiments, a subject antibody is incorporated into a liposome.

A subject anti-SFRP2 antibody can be modified to include a moiety that modifies cellular uptake relative to unconjugated material. The modified antibody may exhibit increased cellular uptake relative to unconjugated material. In alternative embodiments, the modified antibody exhibits decreased cellular uptake relative to unmodified antibody. In this aspect, the efficiency of cellular uptake can be increased or decreased by linking to peptides or proteins that facilitate endocytosis. For example, a given antibody can be linked to a ligand for a target receptor or large molecule that is more easily engulfed by endocytotic mechanisms, such as another antibody. The antibody or other ligand can then be internalized by endocytosis and the payload released by acid hydrolysis or enzymatic activity when the endocytotic vesicle fuses with lysosomes. As such, the conjugate may be one that increases endocytosis relative to unconjugated antibody. To decrease cellular uptake, the modified antibody can include a ligand that retains the antibody on the surface of a cell, which can be useful as a control for cellular uptake, or in some instances decrease uptake in one cell type while increasing it in others.

A subject anti-SFRP2 antibody can comprise one or more moieties, which moieties may be linked (e.g., covalently or non-covalently linked) to the anti-SFRP2 antibody, either directly or via a linker, e.g. a flexible linker. For example, where a subject anti-SFRP2 antibody is a fusion protein comprising an anti-SFRP2 antibody and a heterologous fusion partner polypeptide, the heterologous fusion partner can be linked to the anti-SFRP2 antibody via a linker.

Linkers suitable for use in attaching a moiety to a subject anti-SFRP2 antibody include “flexible linkers”. If present, the linker molecules are generally of sufficient length to permit the anti-SFRP2 antibody and a linked carrier to allow some flexible movement between the anti-SFRP2 antibody and the carrier. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

Suitable linkers can be readily selected and can be of any of a variety of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, GSGGSₙ (SEQ ID NO: 16) and GGGSₙ (SEQ ID NO: 17), where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGSG (SEQ ID NO:18), GGSGG (SEQ ID NO:19), GSGSG (SEQ ID NO: 20), GSGGG (SEQ ID NO: 21), GGGSG (SEQ ID NO: 22), GSSSG (SEQ ID NO: 23), and the like. The ordinarily skilled artisan will recognize that design of a peptide conjugated to any elements described above can include linkers that are all or partially flexible, such that the linker can include a flexible linker as well as one or more portions that confer less flexible structure.

SFRP2

An SFRP2 polypeptide can be targeted in order to regulate levels of glucose and insulin in a subject. Methods and compositions targeting an SFRP2 polypeptide find use in treating and/or preventing aberrant levels of glucose and insulin, even if the subject is on a high-fat diet or has been on a high-fat diet.

“SFRP2 polypeptide” encompasses naturally-occurring full-length and/or fragments of an amino acid sequence of a SFRP2 polypeptide and homologues from different species, as well as SFRP2 polypeptides comprising an amino acid sequence that is substantially similar to the amino acid sequence of a naturally-occurring SFRP2 polypeptide; and use of such proteins in preparation of formulation for therapy and in methods of treating glucose imbalance in a patient. Exemplary embodiments of such are described below.

“SFRP2” (secreted frizzled-related protein 2), as targeted in the method of the present disclosure, is also known as SARP1, FKSG12, unq361, or PRO697″. SFRP2 encompasses murine and human variants that are encoded by the SFRP2 gene or a gene homologous to SFRP2.

SFRP2 refers to SFRP2 proteins or SFRP2 DNA sequences, which encompass their naturally occurring isoforms and/or allelic/splice variants. A SFRP2 protein also refers to proteins that have one or more alteration in the amino acid residues (e.g. at locations that are not conserved across variants and/or species) while retaining the conserved domains and having the same biological activity as the naturally-occurring SFRP2. SFRP2 also encompasses nucleic acid sequences that vary in one or more bases from a naturally-occurring DNA sequence but still translate into an amino acid sequence that correspond to the a naturally-occurring protein due to degeneracy of the genetic code. For example, SFRP2 may also refer to those that differ from the naturally-occurring sequences of SFRP2 by one or more conservative substitutions and/or tags and/or conjugates.

Proteins targeted by the method of the present disclosure can contain a contiguous amino acid residues of a length derived from SFRP2. A sufficient length of contiguous amino acid residues may vary depending on the specific naturally-occurring amino acid sequence from which the protein is derived. For example, the protein may be at least 100 amino acids to 150 amino acid residues in length, at least 150 amino acids to 200 amino acid residues in length, or at least 220 amino acids up to the full-length protein (e.g., 250 amino acids, 282 amino acids, 290 amino acids). For example, the protein may be of about 294 amino acid residues in length when derived from a human SFRP2 protein, or of about 292 amino acid residues in length when derived from a mouse SFRP2 protein.

A protein containing an amino acid sequence that is substantially similar to the amino acid sequence of a SFRP2 polypeptide includes a polypeptide comprising an amino acid sequence having at least about 79%, at least about 80%, at least about 85%, at least about 90%, at least about 96%, at least about 97%, at least about 98%, or at least about 99%, amino acid sequence identity to a contiguous stretch of from about 100 amino acids (aa) to about 150 aa, from about 150 aa to about 200 aa, from about 200 aa to about 250 aa, from about 250 aa up to the full length of a naturally occurring SFRP2 polypeptide. For example, a SFRP2 polypeptide suitable for use in a subject method can comprise an amino acid sequence having at least about 79%, at least about 80%, at least about 85%, at least about 90%, at least about 95%, at least about 96%, at least about 97%, at least about 98%, or at least about 99%, amino acid sequence identity to a contiguous stretch of from about 100 amino acids (aa) to about 150 aa, from about 150 aa to about 200 aa, from about 200 aa to about 250 aa, from about 250 aa up to the full length, of the human SFRP2 polypeptide amino acid sequence depicted in FIG. 6.

The protein may lack at least 5, at least 10, up to at least 50 or more aa relative to a naturally-occurring full-length SFRP2 polypeptide. For example, the protein may not contain the signal sequence of based on the amino acid sequence of a naturally-occurring SFRP2 polypeptide. The protein may also contain the same or similar glycosylation pattern as those of a naturally-occurring SFRP2 polypeptide, may contain no glycosylation, or may contain the glycosylation pattern of host cells used to produce the protein.

Many DNA and protein sequences of SFRP2 are known in the art and certain sequences are discussed later below.

The proteins targeted in the method of the present disclosure include those containing contiguous amino acid sequences of any naturally-occurring SFRP2, as well as those having 1, 2, 3, 4, 5, 6, 7, 8, 9, or 10 usually no more than 20, 10, or 5 amino acid substitutions, where the substitution is usually a conservative amino acid substitution. By “conservative amino acid substitution” generally refers to substitution of amino acid residues within the following groups:

1) L, I, M, V, F;

2) R, K;

3) F, Y, H, W, R;

4) G, A, T, S;

5) Q, N; and

6) D, E.

Conservative amino acid substitutions in the context of a peptide or a protein disclosed herein are selected so as to preserve putative activity of the protein. Such presentation may be preserved by substituting with an amino acid with a side chain of similar acidity, basicity, charge, polarity, or size to the side chain of the amino acid being replaced. Guidance for substitutions, insertion, or deletion may be based on alignments of amino acid sequences of different variant proteins or proteins from different species. For example, according to the alignment shown in FIG. 6, at certain residue positions that are fully conserved (*), substitution, deletion or insertion may not be allowed while at other positions where one or more residues are not conserved, an amino acid change can be tolerated. Residues that are semi-conserved (. or :) may tolerate changes that preserve charge, polarity, and/or size.

The present disclosure provides methods and compositions for targeting any of the SFRP2 polypeptides described above. For screening and characterization purposes, the protein may be isolated from a natural source, e.g., is in an environment other than its naturally-occurring environment. The subject protein may also be recombinantly made, e.g., in a genetically modified host cell (e.g., bacteria; yeast; Pichia; insect cells; and the like), where the genetically modified host cell is genetically modified with a nucleic acid comprising a nucleotide sequence encoding the subject protein. The subject protein encompasses synthetic polypeptides, e.g., a subject synthetic polypeptide is synthesized chemically in a laboratory (e.g., by cell-free chemical synthesis). Methods of productions are described in more detail below.

Nucleic Acid and Protein Sequences

The polypeptide to be targeted in the subject methods may be generated using recombinant techniques to manipulate nucleic acids of different SFRP2 known in the art to provide constructs encoding a protein of interest. It will be appreciated that, provided an amino acid sequence, the ordinarily skilled artisan will immediately recognize a variety of different nucleic acids encoding such amino acid sequence in view of the knowledge of the genetic code.

For production of an SFRP2 protein derived from naturally-occurring polypeptides, it is noted that nucleic acids encoding a variety of different SFRP2 polypeptides are known and available in the art. Nucleic acid (and amino acid sequences) for various SFRP2 are also provided in GenBank as accession nos.: 1) Homo sapiens: amino acid sequence NP_{—}003004.1; nucleotide sequence: NM_{—}003013.2; 2) Mus musculus: amino acid sequence NP_{—}033170.1; nucleotide sequence NM_{—}009144.1; 3) Bos taurus: amino acid sequence NP_{—}001029565; nucleotide sequence NM_{—}001034393; 4) Canis lupus familiaris: amino acid sequence NP_{—}001002987.1; nucleotide sequence NM_{—}001002987.1; 5) Rattus norvegicus: amino acid sequence NP_{—}001094170.1; nucleotide sequence NM_{—}001100700.1; 6) Gallus gallus: amino acid sequence NP_{—}990104.1; nucleotide sequence NM_{—}204773.1. Exemplary amino acid sequences are depicted in FIG. 6. Several sequences and further information on the nucleic acid and protein sequences can also be found in the Example section below.

It will be appreciated that the nucleotide sequences encoding the protein may be modified so as to optimize the codon usage to facilitate expression in a host cell of interest (e.g., Escherichia coli, and the like). Methods for production of codon optimized sequences are known in the art.

Methods of Production

SFRP polypeptides and anti-SFRP2 antibodies can be produced by any suitable method, including recombinant and non-recombinant methods (e.g., chemical synthesis).

Where a polypeptide is chemically synthesized, the synthesis may proceed via liquid-phase or solid-phase. Solid-phase synthesis (SPPS) allows the incorporation of unnatural amino acids, peptide/protein backbone modification. Various forms of SPPS, such as Fmoc and Boc, are available for synthesizing peptides of the present invention. Details of the chemical synthesis are known in the art (e.g. Ganesan A. 2006 Mini Rev. Med Chem. 6:3-10 and Camarero J A et al. 2005 Protein Pept Lett. 12:723-8). Briefly, small insoluble, porous beads are treated with functional units on which peptide chains are built. After repeated cycling of coupling/deprotection, the free N-terminal amine of a solid-phase attached peptide is coupled to a single N-protected amino acid unit. This unit is then deprotected, revealing a new N-terminal amine to which a further amino acid may be attached. The peptide remains immobilized on the solid-phase and undergoes a filtration process before being cleaved off.

Where the SFRP2 protein and/or anti-SFRP2 antibody is produced using recombinant techniques, the proteins and/or antibody may be produced as an intracellular protein or as an secreted protein, using any suitable construct and any suitable host cell, which can be a prokaryotic or eukaryotic cell, such as a bacterial (e.g. E. coli) or a yeast host cell, respectively.

Other examples of eukaryotic cells that may be used as host cells include insect cells, mammalian cells, and/or plant cells. Where mammalian host cells are used, the cells may include one or more of the following: human cells (e.g. HeLa, 293, H9 and Jurkat cells); mouse cells (e.g., NIH3T3, L cells, and C127 cells); primate cells (e.g. Cos 1, Cos 7 and CV1) and hamster cells (e.g., Chinese hamster ovary (CHO) cells).

A wide range of host-vector systems suitable for the expression of the subject protein and/or antibody may be employed according standard procedures known in the art. See for example, Sambrook et al. 1989 Current Protocols in Molecular Biology Cold Spring Harbor Press, New York and Ausubel et al. 1995 Current Protocols in Molecular Biology, Eds. Wiley and Sons.

Methods for introduction of genetic material into host cells include, for example, transformation, electroporation, conjugation, calcium phosphate methods and the like. The method for transfer can be selected so as to provide for stable expression of the introduced SFRP2-encoding nucleic acid. The polypeptide-encoding nucleic acid can be provided as an inheritable episomal element (e.g., plasmid) or can be genomically integrated. A variety of appropriate vectors for use in production of a polypeptide of interest are available commercially.

Vectors can provide for extrachromosomal maintenance in a host cell or can provide for integration into the host cell genome. The expression vector provides transcriptional and translational regulatory sequences, and may provide for inducible or constitutive expression, where the coding region is operably linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, and the like).

Expression constructs generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition the expression construct may contain a selectable marker gene to allow the selection of transformed host cells. Selectable genes are well known in the art and will vary with the host cell used.

Isolation and purification of a protein and/or antibody can be accomplished according to methods known in the art. For example, a protein can be isolated from a lysate of cells genetically modified to express the protein constitutively and/or upon induction, or from a synthetic reaction mixture, by immunoaffinity purification, which generally involves contacting the sample with an anti-protein antibody, washing to remove non-specifically bound material, and eluting the specifically bound protein. The isolated protein can be further purified by dialysis and other methods normally employed in protein purification methods. In one embodiment, the protein may be isolated using metal chelate chromatography methods. Protein of the present disclosure may contain modifications to facilitate isolation.

The subject proteins and/or antibody may be prepared in substantially pure or isolated form (e.g., free from other polypeptides). The protein can present in a composition that is enriched for the polypeptide relative to other components that may be present (e.g., other polypeptides or other host cell components). Purified protein may be provided such that the protein is present in a composition that is substantially free of other expressed proteins, e.g., less than 90%, usually less than 60% and more usually less than 50% of the composition is made up of other expressed proteins.

Antibody Production

A subject anti-SFRP2 antibody can be prepared using a wide variety of techniques known in the art including the use of hybridoma, recombinant, and phage display technologies, or a combination thereof. For example, a subject anti-SFRP2 antibody may be made and isolated using methods of phage display. A subject anti-SFRP2 antibody may also be isolated from sera of an animal host immunized with an immunogenic composition containing an SFRP2 protein, which encompasses whole proteins and fragments thereof.

The antigen that coats the wells for phage display panning or the immunogenic composition used to elicit the antibody of the present disclosure may contain an aggregate of one or more SFRP2 polypeptides, as described above. The method may involve exposing antigens to an aggregating condition so as to form an aggregate. Thus the methods of production described may further include a step of forming an aggregate of the isolated antigens. Examples of the aggregating conditions include heating, addition of an excipient that facilitates aggregation, and the like.

Antigens used to coat the wells for phage panning or to elicit a subject anti-SFRP2 antibody may be conjugated to another molecule. For example, the antigen can be conjugated to a second molecule such as a peptide, polypeptide, lipid, carbohydrate and the like that aids in solubility, storage or other handling properties, cell permeability, half-life, controls release and/or distribution such as by targeting a particular cell (e.g., neurons, leucocytes etc.) or cellular location (e.g., lysosome, endosome, mitochondria etc.), tissue or other bodily location (e.g., blood, neural tissue, particular organs etc.).

A particular embodiment of an antigen conjugated to a second molecule is where the second molecule is an immunomodulator. “Immunomodulator” is a molecule that directly or indirectly modifies an immune response. A specific class of immunomodulators includes those that stimulate or aid in the stimulation of an immunological response. Non-limiting examples include antigens and antigen carriers such as a toxin or derivative thereof, including tetanus toxoid.

Phage Display

Phage display can be used for the high-throughput screening of protein interactions. Phages may be utilized to display antigen-binding domains expressed from a repertoire or combinatorial antibody library (e.g., human or murine). Phage expressing an antigen binding domain that binds SFRP2 can be selected or identified with SFRP2, e.g., using labeled SFRP2 or SFRP2 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene III or gene VIII protein. Details of the methods are set forth, for example, in Nieri P et al. (2009) Curr Med Chem 16:753:79. In another example, ribosomal display can be used to replace bacteriophage as the display platform. Cell surface libraries may be screened for antibodies. Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art.

Immunization and Antibody Production

The method of eliciting antibodies in a host animal involves administering an effective amount of SFRP2 as antigens described above to the host animal (i.e., a suitable mammal such as a mouse, rabbit or guinea pig, or a suitable avian, such as a chicken) to elicit production of an antibody that specifically binds and inhibit SFRP2. Methods of immunizing animal, including the adjuvants used, booster schedules, sites of injection, suitable animals, etc. are well understood in the art, e.g., Harlow et al. (Antibodies: A Laboratory Manual, First Edition (1988) Cold spring Harbor, N.Y.), and administration of living cells to animals has been described for several mammals and birds. Next, a population of antibody producing cells is generated. The population of cells is produced using hybridoma methods that are well known to one of skill in the art (see, e.g., Harlow Antibodies: A Laboratory Manual, First Edition (1988) Cold Spring Harbor, N.Y.). Cells are fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoribosyl transferase (HGPRT). These deficiencies allow selection for fused cells according to their ability to grow on, for example, hypoxanthine-aminopterin-thymidine (HAT) medium. In alternative embodiments, populations of cells expressing monoclonal antibodies may be made using phage display methods.

Anti-SFRP2 antibodies, including antigen binding fragments of anti-SFRP2 antibodies, may also be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library can be constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Phage Panning and Screening

Once the population of antibody-producing cells or phages is produced, the antibodies are screened using one or a combination of a variety of assays. In general, these assays are functional assays, and may be grouped as follows: assays that detect an antibody's binding affinity or specificity, and assays that detect the ability of an antibody to initialize or inhibit a process.

For example, the antigen (e.g. SFRP2) is coupled to beads or wells or other solid support and incubated with phage displaying the antibody of interest. After washings, bound phage is then recovered by inoculation of log phase E. coli cells. The cells are grown and expanded with helper phage. Steps are repeated for the amplification of tightly bound phages. The phage-infected E. coli colonies after several round of enrichment are harvested and Fab antibodies are purified from the periplasmic fractions. The purified antibodies are then analyzed in accordance with methods known in the art. Certain exemplary examples are detailed below.

The population of antibody isolated from phage-infected cells or hybridomas is further analyzed and/or screened for binding to a single antigen (i.e., antigens that are not mixed with other antigens of the plurality of antigens) of the plurality of antigens in vitro or in situ (e.g. on cells). Immunospecific binding may be carried out according to methods routine and known in the art. The immunoassays which can be used include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. See, e.g., Ausubel et al, eds, 1994, Current Protocols in Molecular Biology, Vol. 1, John Wiley & Sons, Inc., New York, which is incorporated by reference herein in its entirety.

In addition to binding assays, the cells and antibodies may be screened based on the ability of the antibody in the supernatant to perform a specific function (e.g. modulation of Wnt signaling pathways).

A subject anti-SFPR2 antibody may also be screened in vivo. The method involves administering a subject antibody to an animal model for a disease or condition and determining the effect of the antibody on the disease or condition of the model animal. In vivo assays of the invention include controls, where suitable controls include a sample in the absence of the antibody. Generally, a plurality of assay mixtures is run in parallel with different antibody concentrations to obtain a differential response to the various concentrations. Typically, one of these concentrations serves as a negative control, i.e., at zero concentration or below the level of detection.

A monoclonal antibody of interest is one that modulates, i.e., reduces or increases a symptom of the animal model disease or condition by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the antibody. In general, a monoclonal antibody of interest will cause a subject animal to be more similar to an equivalent animal that is not suffering from the disease or condition. Antibodies that have therapeutic value that have been identified using the methods and compositions of the invention are termed “therapeutic” antibodies.

Selected monoclonal antibodies of interest can be expanded in vitro, using routine tissue culture methods, or in vivo, using mammalian subjects. For example, pristane-primed mice can be inoculated with log phase hybridoma cells in PBS for ascites production. Ascites fluid can be stored at −70° C. prior to further purification.

Nucleic Acid Encoding the Antibody

Cell expressing a monoclonal antibody of interest contains the immunoglobulin heavy and light chain-encoding expression cassettes. As such, the nucleic acids encoding the monoclonal antibody of interest may be identified. Accordingly, the subject nucleic acids may be identified by a variety of methods known to one of skill in the art. Similar methods are used to identify host cell cultures in monoclonal antibody production using hybridoma technology (Harlow et al., Antibodies: A Laboratory Manual, First Edition (1988) Cold spring Harbor, N.Y.), and rely on an “addressable” host cell and an “addressable” monoclonal antibody, such that once a monoclonal antibody of interest is identified, a host cell address may be determined and the nucleic acid encoding the antibody of interested isolated from the cell.

The nucleic acids encoding a monoclonal antibody of interest may be recovered, characterized and manipulated from a cell expressing the antibody using techniques familiar to one of skill in the art (Ausubel, et al, Short Protocols in Molecular Biology, 3rd ed., Wiley & Sons, (1995) and Sambrook, et al, Molecular Cloning: A Laboratory Manual, Third Edition, (2001) Cold Spring Harbor, N.Y.).

Compositions

The present disclosure provides compositions comprising an anti-SFRP2 antibody, which may be administered to a subject in need of restoring glucose homeostasis.

A subject antibody composition can contain, in addition to a subject antibody, one or more of: a salt, e.g., NaCl, MgCl2, KCl, MgSO4, etc.; a buffering agent, e.g., a Tris buffer, N-(2-Hydroxyethyl)piperazine-N′-(2-ethanesulfonic acid) (HEPES),2-(N-Morpholino)ethanesulfonic acid (MES),2-(N-Morpholino)ethanesulfonic acid sodium salt (MES),3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

Compositions comprising a subject antibody may include a buffer, which is selected according to the desired use of the protein, and may also include other substances appropriate to the intended use. Those skilled in the art can readily select an appropriate buffer, a wide variety of which are known in the art, suitable for an intended use.

The composition may comprise a pharmaceutically acceptable excipient, diluent, or carrier, a variety of which are known in the art and need not be discussed in detail herein. Pharmaceutically acceptable excipients have been amply described in a variety of publications, including, for example, “Remington: The Science and Practice of Pharmacy”, 19th Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

A subject pharmaceutical composition can comprise an anti-SFRP2 antibody, and a pharmaceutically acceptable excipient, diluent, or carrier. In some cases, a subject pharmaceutical composition will be suitable for injection into a subject, e.g., will be sterile. For example, in some embodiments, a subject pharmaceutical composition will be suitable for injection into a human subject, e.g., where the composition is sterile and is free of detectable pyrogens and/or other toxins.

A subject antibody composition may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include aqueous solution, powder form, granules, tablets, pills, suppositories, capsules, suspensions, sprays, suppositories, and the like. The composition may be formulated according to the different routes of administration described later below.

Where the antibody is administered as an injectable (e.g. subcutaneously, intraperitoneally, and/or intravenous) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The antibody-containing formulations may also be provided so as to enhance serum half-life of the subject protein following administration. For example, the antibody may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include isotonic sterile injection solutions. A formulation can include one or more of anti-oxidants, bacteriostats, and solutes that render the formulation isotonic with the blood of the intended recipient. A formulation can include one or more of suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The concentration of a subject antibody in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

Patient Populations

The present disclosure provides a method to treat a patient suffering from hyperglycemia, hyperinsulinemia, and/or glucose intolerance. Such conditions are also commonly associated with many other glucose metabolism disorders. As such, patients having glucose metabolism disorders can be candidates for therapy according to the subject methods.

The phrase “glucose metabolism disorder” encompasses any disorder characterized by a clinical symptom or a combination of clinical symptoms that are associated with an elevated level of glucose and/or an elevated level of insulin in a subject relative to a healthy individual. Symptoms that are associated with an elevated level of glucose and/or an elevated level of insulin in a subject, relative to a healthy individual, can include, e.g., frequent urination, unusual thirst, extreme hunger, extreme fatigue and irritability, blurred vision and tingling/numbness in the hands/feet. Elevated levels of glucose and/or insulin may be manifested in the following disorders and/or conditions: type II diabetes (e.g. insulin-resistance diabetes), gestational diabetes, insulin resistance, impaired glucose tolerance, hyperinsulinemia, impaired glucose metabolism, pre-diabetes, metabolic disorders (such as metabolic syndrome which is also referred to as syndrome X), obesity, obesity-related disorder. One indication of obesity is a high Body Mass Index (BMI). An adult having a BMI in the range of 18.5 to 24.9 is considered to have a normal weight; an adult who has a BMI between 25 and 29.9 kg/m2 may be considered overweight (pre-obese); an adult who has a BMI of 30 kg/m2 or higher may be considered obese.

An example of a suitable patient may be one who is hyperglycemic and/or hyperinsulinemic and who is also diagnosed with diabetes mellitus (e.g. Type II diabetes). “Diabetes” refers to a progressive disease of carbohydrate metabolism involving inadequate production or utilization of insulin and is characterized by hyperglycemia and glycosuria.

“Hyperglycemia”, as used herein, is a condition in which an elevated amount of glucose circulates in the blood plasma relative to a healthy individual and can be diagnosed using methods known in the art. For example, hyperglycemia can be diagnosed as having a fasting blood glucose level between 5.6 to 7 mM (pre-diabetes), or greater than 7 mM (diabetes).

“Hyperinsulinemia”, as used herein, is a condition in which there are elevated levels of circulating insulin while blood glucose levels may either be elevated or remain normal. Hyperinsulinemia can be caused by insulin resistance which is associated with dyslipidemia such as high triglycerides, high cholesterol, high low-density lipoprotein (LDL) and low high-density lipoprotein (HDL), high uric acids, polycystic ovary syndrome, type II diabetes and obesity. Hyperinsulinemia can be diagnosed as having a plasma insulin level higher than about 2 μU/mL.

A patient having any of the above disorders may be a suitable candidate in need of a therapy in accordance with the present method so as to receive treatment for hyperglycemia, hyperinsulinemia, and/or glucose intolerance. Administering the subject protein in such an individual can restore glucose homeostasis and may also decrease one or more of symptoms associated with the disorder.

Candidates for treatment using the subject method may be determined using diagnostic methods known in the art, e.g. by assaying plasma glucose and/or insulin levels. Candidates for treatment include those who have exhibited or are exhibiting higher than normal levels of plasma glucose/insulin. Such patients include patients who have a fasting blood glucose concentration (where the test is done after 8 to 10 hour fast) of higher than about 100 mg/dL, e.g., higher than about 110 mg/dL, higher than about 120 mg/dL, about 150 mg/dL up to about 200 mg/dL or more. Individuals suitable to be treated also include those who have a 2 hour postprandial blood glucose concentration or a concentration after a glucose tolerance test (e.g. 2 hours after ingestion of a glucose-rich drink), in which the concentration is higher than about 140 mg/dL, e.g., higher than about 150 mg/dL up to 200 mg/dL or more. Glucose concentration may also be presented in the units of mmol/L, which can be acquired by dividing mg/dL by a factor of 18.

Methods

The subject method involves administering an anti-SFRP2 antibody to a subject who has hyperglycemia, hyperinsulinemia, and/or glucose intolerance. The methods of the present disclosure include administering one or more anti-SFRP2 antibodies in the context of a variety of conditions including glucose metabolism disorders, including the examples above (in both prevention and post-diagnosis therapy).

Subjects having, suspected of having, or at risk of developing a glucose metabolism disorder are contemplated for therapy and diagnosis described herein.

By “treatment” is meant that at least an amelioration of the symptoms associated with the condition afflicting the host is achieved, where amelioration refers to at least a reduction in the magnitude of a parameter, e.g. symptom, associated with the condition being treated. As such, treatment includes situations where the condition, or at least symptoms associated therewith, are reduced or avoided. Thus treatment includes: (i) prevention, that is, reducing the risk of development of clinical symptoms, including causing the clinical symptoms not to develop, e.g., preventing disease progression to a harmful or otherwise undesired state; (ii) inhibition, that is, arresting the development or further development of clinical symptoms, e.g., mitigating or completely inhibiting an active disease (e.g., so as to decrease level of insulin and/or glucose in the bloodstream, to increase glucose tolerance so as to minimize fluctuation of glucose levels, and/or so as to protect against diseases caused by disruption of glucose homeostasis).

In the methods of the present disclosure, antibody compositions described herein can be administered to a subject (e.g. a human patient) to, for example, achieve and/or maintain glucose homeostasis, e.g., to reduce glucose level in the bloodstream and/or to reduce insulin level to a range found in a healthy individual. Subjects for treatment include those having a glucose metabolism disorder as described herein. For example, a subject antibody composition finds use in facilitating glucose homeostasis in subjects with a glucose metabolism disorder resulting from obesity.

The methods relating to disorders of the glucose metabolism contemplated herein include, for example, use of an antibody described above for therapy alone or in combination with other types of therapy. The method involves administering to a subject a subject antibody (e.g. subcutaneously or intravenously). As noted above, the methods are useful in the context of treating or preventing a wide variety of disorders related to glucose metabolism.

Routes of Administration

In practicing the methods, routes of administration (path by which a subject protein is brought into a subject) may vary. A subject antibody above can be delivered by a route that provides for delivery of the protein to the bloodstream (e.g., by parenteral administration, such as intravenous administration, intramuscular administration, and/or subcutaneous administration). Injection can be used to accomplish parenteral administration.

Combination Therapy

Any of a wide variety of therapies directed to regulating glucose metabolism, and any glucose metabolism disorders, and/or obesity, for example, can be combined in a composition or therapeutic method with the subject antibody. A subject antibody can also be administered in combination with a modified diet and/or exercise regimen to promote weight loss.

“Combination” as used herein is meant to include therapies that can be administered separately, e.g. formulated separately for separate administration (e.g., as may be provided in a kit), or undertaken as a separate regime (as in exercise and diet modifications), as well as for administration in a single formulation (i.e., “co-formulated”). Examples of agents that may be provided in a combination therapy include those that are normally administered to subjects suffering from symptoms of hyperglycemia, hyperinsulinemia, glucose intolerance, and disorders associated those conditions. Examples of agents that may be provided in a combination therapy include those that promote weight loss.

Where a subject antibody is administered in combination with one or more other therapies, the combination can be administered anywhere from simultaneously to up to 5 hours or more, e.g., 10 hours, 15 hours, 20 hours or more, prior to or after administration of a subject protein. In certain embodiments, a subject antibody and other therapeutic intervention are administered or applied sequentially, e.g., where a subject antibody is administered before or after another therapeutic treatment. In yet other embodiments, a subject antibody and other therapy are administered simultaneously, e.g., where a subject antibody and a second therapy are administered at the same time, e.g., when the second therapy is a drug it can be administered along with a subject antibody as two separate formulations or combined into a single composition that is administered to the subject. Regardless of whether administered sequentially or simultaneously, as illustrated above, the treatments are considered to be administered together or in combination for purposes of the present disclosure.

Additional standard therapeutics for glucose metabolism disorders that may or may not be administered in conjunction with a subject protein, include but not limited to any of the combination therapies described above, hormonal therapy, immunotherapy, chemotherapeutic agents and surgery.

Dosages

In the methods, a therapeutically effective amount of a subject antibody is administered to a subject in need thereof. For example, a subject antibody causes the level of plasma glucose and/or insulin to return to a normal level relative to a healthy individual when the subject antibody is delivered to the bloodstream in an effective amount to a patient who previously did not have a normal level of glucose/insulin relative to a healthy individual prior to being treated. The amount administered varies depending upon the goal of the administration, the health and physical condition of the individual to be treated, age, the degree of resolution desired, the formulation of a subject antibody, the activity of the subject antibody employed, the treating clinician's assessment of the medical situation, the condition of the subject, and the body weight of the subject, as well as the severity of the dysregulation of glucose/insulin and the stage of the disease, and other relevant factors. The size of the dose will also be determined by the existence, nature, and extent of any adverse side-effects that might accompany the administration of a particular antibody.

It is expected that the amount will fall in a relatively broad range that can be determined through routine trials. For example, the amount of subject antibody employed to restore glucose homeostasis is not more than about the amount that could otherwise be irreversibly toxic to the subject (i.e., maximum tolerated dose). In other cases, the amount is around or even well below the toxic threshold, but still in an effective concentration range, or even as low as threshold dose.

Also, suitable doses and dosage regimens can be determined by comparisons to indicators of glucose metabolism. Such dosages include dosages which result in the stabilized levels of glucose and insulin, for example, comparable to a healthy individual, without significant side effects. Dosage treatment may be a single dose schedule or a multiple dose schedule (e.g., including ramp and maintenance doses). As indicated below, a subject composition may be administered in conjunction with other agents, and thus doses and regimens can vary in this context as well to suit the needs of the subject.

Individual doses are typically not less than an amount required to produce a measurable effect on the subject, and may be determined based on the pharmacokinetics and pharmacology for absorption, distribution, metabolism, and excretion (“ADME”) of the subject antibody or its by-products, and thus based on the disposition of the composition within the subject. This includes consideration of the route of administration as well as dosage amount, which can be adjusted for enteral (applied via digestive tract for systemic or local effects when retained in part of the digestive tract) or parenteral (applied by routes other than the digestive tract for systemic or local effects) applications. For instance, administration of a subject antibody is typically via injection and often intravenous, intramuscular, or a combination thereof.

By “therapeutically effective amount” is meant that the administration of that amount to an individual, either in a single dose, as part of a series of the same or different antibody compositions, is effective to help restore homeostasis of glucose metabolism as assessed by glucose and/or insulin levels in a subject. As noted above, the therapeutically effective amount can be adjusted in connection with dosing regimen and diagnostic analysis of the subject's condition (e.g., monitoring for the levels of glucose and/or insulin in the plasma) and the like.

As an example, the effective amount of a dose or dosing regimen can be gauged from the ED₅₀ of a protein for inducing an action that leads to clearing glucose from the bloodstream or lowering of insulin levels. By “ED₅₀” (effective dosage) is the intended dosage which induces a response halfway between the baseline and maximum after some specified exposure time. The ED₅₀ of a graded dose response curve therefore represents the concentration of a subject protein where 50% of its maximal effect is observed. ED₅₀ may be determined by in vivo studies (e.g. animal models) using methods known in the art.

An effective amount may not be more than 100× the calculated ED₅₀. For instance, the amount of protein that is administered is less than about 100×, less than about 50×, less than about 40×, 35×, 30×, or 25× and many embodiments less than about 20×, less than about 15× and even less than about 10×, 9×, 9×, 7×, 6×, 5×, 4×, 3×, 2× or 1× than the calculated ED₅₀. In one embodiment, the effective amount is about 1× to 30× of the calculated ED₅₀, and sometimes about 1× to 20×, or about 1× to 10× of the calculated ED₅₀. In other embodiments, the effective amount is the same as the calculated ED₅₀, and in certain embodiments the effective amount is an amount that is more than the calculated ED₅₀.

An effective amount of a subject antibody may also be an amount that is effective, when administered in one or more doses, to reduce in an individual a level of plasma glucose and/or plasma insulin that is elevated relative to that of a healthy individual by at least about 10%, at least about 20%, at least about 25%, at least about 30%, at least about 40%, at least about 50%, at least about 60%, at least about 70%, at least about 80%, or more than 80%, compared to an elevated level of plasma glucose/insulin in the individual not treated with the protein.

Further examples of dose per administration may be at less than 10 less than 2 or less than 1 μg. Dose per administration may also be more than 50 more than 100 more than 300 μg up to 600 μg or more. An example of a range of dosage per weight is about 0.1 μg/kg to about 1 μg/kg, up to about 1 mg/kg or more. Effective amounts and dosage regimen can readily be determined empirically from assays, from safety and escalation and dose range trials, individual clinician-patient relationships, as well as in vitro and in vivo assays known in the art.

The term “unit dosage form,” as used herein, refers to physically discrete units suitable as unitary dosages for human and animal subjects, each unit containing a predetermined quantity of proteins of the present disclosure calculated in an amount sufficient to produce the desired effect in association with a pharmaceutically acceptable diluent, carrier or vehicle. The specifications for the novel unit dosage forms depend on the particular protein employed and the effect to be achieved, and the pharmacodynamics associated with each protein in the host.

Methods of Screening

A screening method of the present disclosure can be employed to screen for a binding agent that down regulates (e.g., inhibits or neutralizes) SFRP2 activity. The method can involve contacting an SFRP2 polypeptide with a candidate agent and detecting binding of the candidate agent with SFRP2. The method may also involve contacting SFRP2 with a candidate agent in the presence or absence of one or more known interacting molecules of SFRP2 and detecting the binding of the candidate agent with SFRP2 and/or the interacting molecules with SFRP2. The method may also involve the use of libraries of constructs encoding antibodies, aptamers, and/or libraries of small molecules to screen for a SFRP2-binding agent. The binding agent may be selected for its potent inhibition of SFRP2 activities, inhibition of the expression of mature SFRP2, and/or inhibition of the binding affinity for SFRP2-interacting proteins (molecules known to participate in the Wnt signaling pathway). The method may be executed according to methods known in the art.

Briefly, SFRP2 (e.g. SFRP2 alone or SFRP2 complexed with its interacting molecules, such as proteins from the Wnt family) is contacted with a candidate agent. The binding of the candidate agent to SFRP2 is measured to see if there is a binding affinity for SFRP2. The ability of the candidate agent to disrupt SFRP2 binding to its interacting molecules (e.g. proteins of the Wnt family) is also assessed. Candidate agents that are effective in disrupting binding of SFRP2 to its interacting molecules are selected to be potential agents to be used in diagnostic and therapeutic compositions and methods of use. Candidate agents that can disrupt binding of SFRP2 to its interacting molecules (e.g. Wnt proteins) encompass those that can decrease the binding affinity of SFRP2 to its interacting partners either competitively or noncompetitively.

SFRP2 that may be used to screen for potential agent include SFRP2 as described previously. Examples of SFRP2 to be used in the subject screening methods include but are not limited to full-length SFRP2, mature SFRP2, fragments of SFRP2, such as a fragment of SFRP2 lacking the membrane anchor, SFRP2 alone or SFRP2 bound to one or more interacting molecules of the Wnt pathways.

In an example of a screening method, SFRP2 may be immobilized on an ELISA plate or on beads through a covalent or non-covalent interaction, such as hydrophobic adsorption, biotin-avidin interaction, and Ni2+-6×His interaction. A population of candidate agents is then incubated with the immobilized SFRP2, washed, and recovered. During selection, the bound candidate is recovered and identified. Multiple successive selection rounds ensure a selection of a candidate that acts as a specific binding agent for SFRP2. Other methods such as surface plasmon resonance, western blot, functional assays (e.g. phosphorylation or dephosphorylation of downstream targets), fluorescence activated cell sorting, etc. can also be used to screen and select for agents that can bind SFRP2, and/or inhibit its interaction to one or more interacting molecules. Other assays known in the art that involve comparing binding and/or activity of SFRP2 in the presence or absence of the candidate agents can be employed.

Candidate SFRP2-binding agents may also be engineered so that the agent contains sites that are known to have affinity for the ligand-binding site (e.g. cysteine rich-domain homologous to the Wnt-binding site).

Kits

Also provided by the present disclosure are kits for using the compositions disclosed herein and for practicing the methods, as described above. The kits may be provided for administration of the subject protein in a subject in need of restoring glucose homeostasis. The kit can include one or more of an SFRP2 polypeptide and/or an anti-SFRP2 antibody disclosed herein, which may be provided in a sterile container, and can be provided in formulation with a suitable pharmaceutically acceptable excipient for administration to a subject. The proteins (e.g., an anti-SFRP2 antibody) can be provided with a formulation that is ready to be used as it is or can be reconstituted to have the desired concentrations. Where the proteins (e.g., an anti-SFRP2 antibody) are provided to be reconstituted by a user, the kit may also provide buffers, pharmaceutically acceptable excipient, and the like, packaged separately from the subject protein. The proteins (e.g., an anti-SFRP2 antibody) of the present kit may be formulated separately or in combination with other drugs. Where an antibody is formulated separately with another drug, a subject kit can include: 1) a first container (e.g., a sterile container) comprising a subject pharmaceutical composition (e.g., a pharmaceutical composition comprising a subject anti-SFRP2 antibody); and 2) a second container (e.g., a sterile container) comprising a second agent (e.g., a second agent that can lower blood glucose levels).

In addition to above-mentioned components, the kits can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

Materials and Methods

The following methods and materials were used in the Examples below.

Animals.

C57BL/6 mice were purchased from the Charles River Laboratory (Wilmington, Mass.). Mice were kept in accordance with welfare guidelines under controlled light (12 hr light and 12 hr dark cycle, dark 6:30 pm-6:30 am), temperature (22±4° C.) and humidity (50%±20%) conditions. They had free access to water (autoclaved distilled water) and were fed ad libitum on a commercial diet (Harlan laboratories, Irradiated 2018 Teklad Global 18% Protein Rodent Diet) containing 17 kcal % fat, 23 kcal % protein and 60 kcal % carbohydrate. Alternatively, mice were maintained on a high-fat diet (D12492, Research Diet, New Brunswick, N.J. USA) containing 60 kcal % fat, 20 kcal % protein and 20 kcal % carbohydrate. All animal studies were approved by the NGM Institutional Animal Care and Use Committee for NGM-5-2008 entitled “Characterization Of Biologics, Compounds And Viral Vectors For Treatment Of Diabetes Using Rodent Model”.

DNA and Amino Acid Sequences.

cDNA of ORF Encoding Murine Sfrp2

(GenBank Accession No. NM_{—}009144.1)

(SEQ ID NO: 24)
ATGCCGCGGGGCCCTGCCTCGCTGCTGCTGCTAGTCCTCGCCTCGCACTG
CTGCCTGGGCTCGGCGCGTGGGCTCTTCCTCTTCGGCCAGCCCGACTTCT
CCTACAAGCGCAGCAACTGCAAGCCCATCCCCGCCAACCTGCAGCTGTGC
CACGGCATCGAGTACCAGAACATGCGGCTGCCCAACCTGCTGGGCCACGA
GACCATGAAGGAGGTGCTGGAGCAGGCGGGCGCCTGGATTCCGCTGGTCA
TGAAGCAGTGCCACCCGGACACCAAGAAGTTCCTGTGCTCGCTCTTCGCC
CCTGTCTGTCTCGACGACCTAGATGAGACCATCCAGCCGTGTCACTCGCT
CTGCGTGCAGGTGAAGGACCGCTGCGCCCCGGTCATGTCCGCCTTCGGCT
TCCCCTGGCCAGACATGCTGGAGTGCGACCGTTTCCCGCAGGACAACGAC
CTCTGCATCCCCCTCGCTAGTAGCGACCACCTCCTGCCGGCCACAGAGGA
AGCTCCCAAGGTGTGTGAAGCCTGCAAAACCAAGAATGAGGACGACAACG
ACATCATGGAAACCCTTTGTAAAAATGACTTCGCACTGAAAATCAAAGTG
AAGGAGATAACGTACATCAACAGAGACACCAAGATCATCCTGGAGACAAA
GAGCAAGACCATTTACAAGCTGAACGGCGTGTCCGAAAGGGACCTGAAGA
AATCCGTGCTGTGGCTCAAAGACAGCCTGCAGTGCACCTGTGAGGAGATG
AACGACATCAACGCTCCGTATCTGGTCATGGGACAGAAGCAGGGCGGCGA
GCTGGTGATCACCTCCGTGAAACGGTGGCAGAAGGGCCAGAGAGAGTTCA
AGCGCATCTCCCGCAGCATCCGCAAGCTGCAATGCTAG.

Protein sequence encoded by the cDNA (GenBank Accession No. NP_{—}033170.1)

(SEQ ID NO: 25)
MPRGPASLLLLVLASHCCLGSARGLFLFGQPDFSYKRSNCKPIPANLQLC
HGIEYQNMRLPNLLGHETMKEVLEQAGAWIPLVMKQCHPDTKKFLCSLFA
PVCLDDLDETIQPCHSLCVQVKDRCAPVMSAFGFPWPDMLECDRFPQDND
LCIPLASSDHLLPATEEAPKVCEACKTKNEDDNDIMETLCKNDFALKIKV
KEITYINRDTKIILETKSKTIYKLNGVSERDLKKSVLWLKDSLQCTCEEM
NDINAPYLVMGQKQGGELVITSVKRWQKGQREFKRISRSIRKLQC.

Sfrp2 open reading frame (ORF) was amplified with polymerase chain reaction (PCR) using recombinant DNA (cDNA) prepared from mouse small intestinal tissue. PCR reagents kits with Phusion high-fidelity DNA polymerase were purchased from New England BioLabs (F-530L, Ipswich, Mass.). The following primers were used: forward PCR primer: 5′ ATGCCGCGGGGCCCTGCCTC (SEQ ID NO: 26) and reverse PCR primer: 5′ CTAGCATTGCAGCTTGCGGAT (SEQ ID NO: 27).

PCR.

The PCR reactions were set up according to manufacturer's instruction, amplified DNA fragment was digested with restriction enzymes Spe I and Not I (the restriction sites were included in the 5′ or 3′ PCR primers, respectively), and the amplification product was then ligated with AAV transgene vectors that had been digested with the same restriction enzymes. The vector used for expression contained a selectable marker and an expression cassette composed of a strong eukaryotic promoter 5′ of a site for insertion of the cloned coding sequence, followed by a 3′ untranslated region and bovine growth hormone polyadenylation tail. The expression construct is also flanked by internal terminal repeats at the 5′ and 3′ ends.

Production and Purification of AAV.

AAV 293 cells (obtained from Agilent Technologies, Santa Clara, Calif.) were cultured in Dulbecco's Modification of Eagle's Medium (DMEM, Mediatech, Inc. Manassas, Va.) supplemented with 10% fetal bovine serum and 1× antibiotic-antimycotic solution (Mediatech, Inc. Manassas, Va.). The cells were plated at 50% density on day 1 in 150 mm cell culture plates and transfected on day 2, using calcium phosphate precipitation method, with the following 3 plasmids (20 μg/plate of each): AAV transgene plasmid, pHelper plasmids (Agilent technologies) and AAV2/9 plasmid (Gao et al (2004) J. Virol. 78:6381). 48 hours after transfection, the cells were scrapped off the plates, pelleted by centrifugation at 3000×g and resuspended in buffer containing 20 mM Tris pH 8.5, 100 mM NaCl and 1 mM MgCl₂. The suspension was frozen in an alcohol dry ice bath and was then thawed in 37° C. water bath. The freeze and thaw cycles were repeated for three times; Benzenase (Sigma-aldrich, St. Louis, Mo.) were added to a final concentration of 50 units/ml; deoxycholate were added to a final concentration of 0.25%. After an incubation at 37° C. for 30 min, cell debris was pelleted by centrifugation at 5000×g for 20 min. Viral particles in the supernatant were purified using a discontinuous iodixanol (Sigma-Aldrich, St. Louis, Mo.) gradient as previously described (Zolotukhin S et al (1999) Gene Ther. 6:973). The viral stock was concentrated using Vivaspin 20 (MW cutoff 100,000 Dalton, Sartorius Stedim Biotech, Aubagne, France) and re-suspended in phosphate buffered saline (PBS) with 10% glycerol and stored at −80° C. To determine the viral genome copy number, 2 μl of viral stock were incubated in 6 μl of solution containing 50 units/ml Benzonase, 50 mM Tris-HCl pH 7.5, 10 mM Mg Cl₂ and 10 mM Ca Cl2 for at 37° C. for 30 minutes.

Afterwards, 15 μl of the solution containing 2 mg/ml of Proteinase K, 0.5% SDS and 25 mM EDTA were added and the mixture was incubated for additional 20 min at 55° C. release viral DNA. Viral DNA was cleaned with mini DNeasy Kit (Qiagen, Valencia, Calif.) and eluted with 40 μl of water. Viral genome copy (GC) was determined by using quantitative PCR.

Viral stock was diluted with PBS to desirable GC/ml. 200 μl of viral working solution was delivered into mice via tail vein injection.

Blood Glucose Assay.

Blood glucose in mouse tail snip was measured using ACCU-CHEK Active test strips read by ACCU-CHEK Active meter (Roche Diagnostics, Indianapolis, Ind.) following manufacturer's instruction.

Serum Insulin Assay.

Whole blood (about 50 μl/mouse) from mouse tail snips was collected into plain capillary tubes (BD Clay Adams SurePrep, Becton Dickenson and Co. Sparks, Md.). Serum and blood cells were separated by spinning the tubes in an Autocrit Utra 3 (Becton Dickinson and Co. Sparks, Md.). Insulin levels in serum were determined using insulin EIA kits (80-Insums-E01, Alpco Diagnostics, Salem, N.H.) by following manufacturer's instruction.

Glucose Tolerance Test (GTT).

Mice fasted for 16 hours received glucose (1 g/kg) in PBS via intra-peritoneal injection. Blood glucose levels were determined as described above at the time points indicated.

Insulin Tolerance Test (ITT).

Mice fasted for 4 hours received 0.75 units/kg of insulin (Humulin R Eli Lilly and Co. Indianapolis, Ind.) via intra-peritoneal injection. Blood glucose was determined as described above.

Statistics.

Statistical analysis was performed with Student's t-Test with 2-tailed distribution.

### Example 1

### Effect of In Vivo Sfrp2 Expression on Blood Glucose Levels in Mice with Diet-Induced Obesity

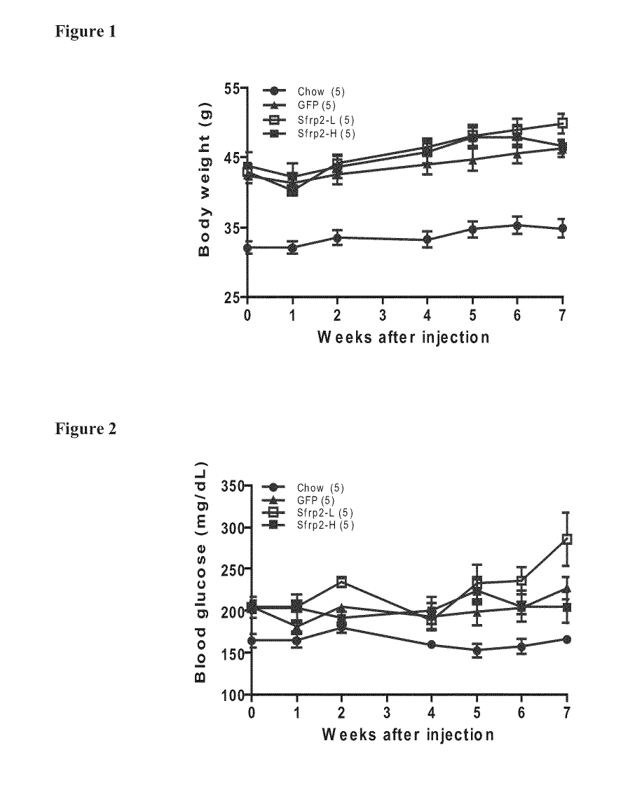

To identify secreted proteins that have an effect on glucose metabolism, selected genes were overexpressed in mice using adeno-associated virus (AAV) as the gene delivery vehicle. The effects of the gene products on glucose metabolism were evaluated in diet-induced obesity (DIO) model. Eight weeks old male C57B/6 mice were subjected to 60% kcal fat diet for eight week before they received a one-time tail vein injection of recombinant AAV (rAAV). Mice body weight (FIG. 1), blood glucose and serum insulin were determined. Glucose tolerance and insulin tolerance tests were also performed to help the assessment of effect of rAAV on glucose clearance and insulin sensitivity.

The ability of murine Sfrp2 to regulate the level of plasma glucose was tested as follows. rAAV expressing Sfrp2 was injected through tail vein into mice that had been on high fat diet for eight weeks. Two weeks after the injection, 4-hour fasting blood glucose levels were determined in tail bleed. In FIG. 2, “Chow” refers to lean mice on chow diet, “GFP” to DIO mice that were injected with 1×10¹² genome copies (“1E+12” “GC”) of rAAV expressing green fluorescent protein, “Sfrp2-L” to mice injected with 3E+11GC of rAAV expressing Sfrp2, and “Sfrp2-H” to mice injected with 1E+12GC of rAAV expressing Sfrp2.

### Example 2

### Effect of Murine Sfrp2 Expression on Serum Insulin Levels in Mice with Diet-Induced Obesity

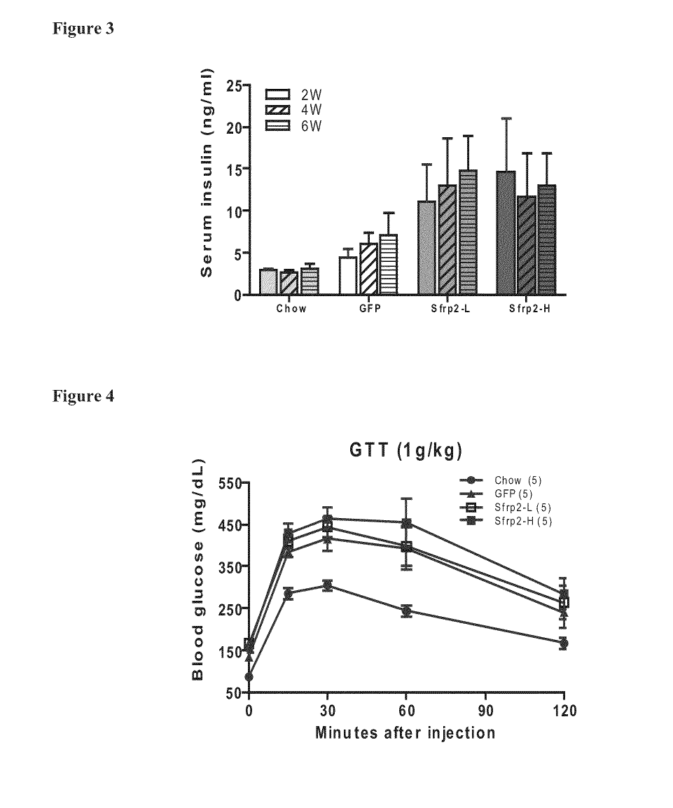

The ability of murine Sfrp2 to affect hyperinsulinemia in mice with diet-induced obesity was tested. rAAV was injected through a tail vein into mice that had been on high fat diet for eight weeks. At the two week and four week time points after the AAV injection, tail blood was collected from mice that had been fasting for four hours, and serum insulin were determined by enzyme-linked immunosorbent assay (ELISA). In FIG. 3, “Chow” refers to lean mice on chow diet; “GFP” to DIO mice that were injected with 1E+12 GC of rAAV expressing green fluorescent protein, “Sfrp2-L” to mice injected with 3E+11 GC of rAAV expressing Sfrp2, and “Sfrp2-H” to mice injected with 1E+12 GC of rAAV expressing Sfrp2. As seen in FIG. 3, recombinant AVV expressing murine Sfrp2 increased the plasma level of insulin in DIO mice.

### Example 3

### Effect of Murine SFRP2 Expression on Glucose Tolerance in Mice with Diet-Induced Obesity

The effect of murine Sfrp2 on glucose tolerance of mice with diet-induced obesity was evaluated as follows. rAAV expressing Sfrp2 was injected through tail vein into mice that had been on high fat diet for eight weeks. Glucose tolerance test was performed three weeks after the AAV injection. Mice fasted overnight received 1 g/kg of glucose in PBS via intraperitoneal injection (i.p.). Blood glucose levels were determined at times indicated. In FIG. 4, “Chow” refers to mice on chow (lean) diet, “GFP” to DIO mice that were injected with 1E+12 GC of rAAV expressing green fluorescent protein, and “Sfrp2-L” to mice injected with 1E+12 GC of rAAV expressing Sfrp2.

### Example 4

### Effect of Murine Sfrp2 Expression on Insulin Tolerance in Mice with Diet-Induced Obesity

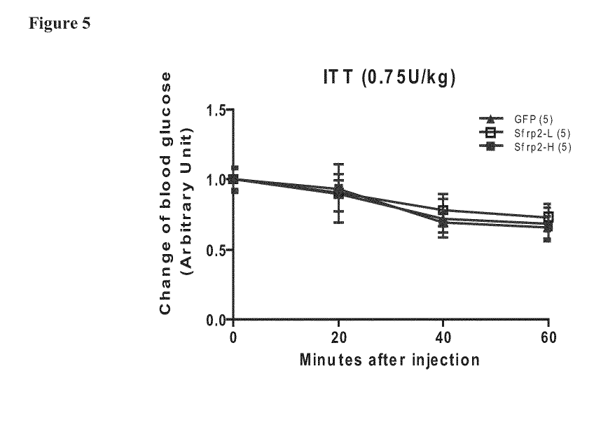

The effect of murine Sfrp2 on insulin sensitivity of mice with diet-induced obesity was evaluated as follows. rAAV expressing Sfrp2 was injected through tail vein into mice that had been on high fat diet for eight weeks. Insulin tolerance test was performed five weeks after the AAV injection. Glucose levels were monitored after an intraperitoneal injection of insulin (0.75 units/kg). Response to insulin was compared among DIO mice injected with AAV expressing Sfrp2, GFP and lean mice by measuring blood glucose levels at times indicated. In FIG. 5, “Chow” refers to mice on chow (lean) diet, “GFP” to DIO mice that were injected with 1E+12 GC of rAAV expressing green fluorescent protein, and “Sfrp2-L” to mice injected with 1E+12 GC of rAAV expressing Sfrp2.

### Example 5

### Cloning of the Human Gene

The cloning of the human gene encoding SFRP2 is carried out by using the PCR method as previously set forth for the cloning of the mouse gene. Briefly, the human SFRP2 gene variant 1 can be cloned out by PCR from cDNA library using the following pair of primers, and then cloned into AAV transgene vector as described above for efficacy evaluation. Forward PCR primer: 5′ ATGCTGCAGGGCCCTGGCTC (SEQ ID NO: 1). Reverse PCR primer: 5′ CTAGCACTGCAGCTTGCGGA (SEQ ID NO: 2).

The nucleic acid sequences, and the encoded amino acid sequence, for human SFRP2 are provided below:

Human SFRP2 ORF

(GenBank Accession No. NM_{—}003013.2)

(SEQ ID NO: 3)
ATGCTGCAGGGCCCTGGCTCGCTGCTGCTGCTCTTCCTCGCCTCGCACTG
CTGCCTGGGCTCGGCGCGCGGGCTCTTCCTCTTTGGCCAGCCCGACTTCT
CCTACAAGCGCAGCAATTGCAAGCCCATCCCTGCCAACCTGCAGCTGTGC
CACGGCATCGAATACCAGAACATGCGGCTGCCCAACCTGCTGGGCCACGA
GACCATGAAGGAGGTGCTGGAGCAGGCCGGCGCTTGGATCCCGCTGGTCA
TGAAGCAGTGCCACCCGGACACCAAGAAGTTCCTGTGCTCGCTCTTCGCC
CCCGTCTGCCTCGATGACCTAGACGAGACCATCCAGCCATGCCACTCGCT
CTGCGTGCAGGTGAAGGACCGCTGCGCCCCGGTCATGTCCGCCTTCGGCT
TCCCCTGGCCCGACATGCTTGAGTGCGACCGTTTCCCCCAGGACAACGAC
CTTTGCATCCCCCTCGCTAGCAGCGACCACCTCCTGCCAGCCACCGAGGA
AGCTCCAAAGGTATGTGAAGCCTGCAAAAATAAAAATGATGATGACAACG
ACATAATGGAAACGCTTTGTAAAAATGATTTTGCACTGAAAATAAAAGTG
AAGGAGATAACCTACATCAACCGAGATACCAAAATCATCCTGGAGACCAA
GAGCAAGACCATTTACAAGCTGAACGGTGTGTCCGAAAGGGACCTGAAGA
AATCGGTGCTGTGGCTCAAAGACAGCTTGCAGTGCACCTGTGAGGAGATG
AACGACATCAACGCGCCCTATCTGGTCATGGGACAGAAACAGGGTGGGGA
GCTGGTGATCACCTCGGTGAAGCGGTGGCAGAAGGGGCAGAGAGAGTTCA
AGCGCATCTCCCGCAGCATCCGCAAGCTGCAGTGCTAG.

Human SFRP2

295 amino acid residues (GenBank Accession No. NP_{—}003004.1).

(SEQ ID NO: 4)
MLQGPGSLLLLFLASHCCLGSARGLFLFGQPDFSYKRSNCKPIPANLQLC
HGIEYQNMRLPNLLGHETMKEVLEQAGAWIPLVMKQCHPDTKKFLCSLFA
PVCLDDLDETIQPCHSLCVQVKDRCAPVMSAFGFPWPDMLECDRFPQDND
LCIPLASSDHLLPATEEAPKVCEACKNKNDDDNDIMETLCKNDFALKIKV
KEITYINRDTKIILETKSKTIYKLNGVSERDLKKSVLWLKDSLQCTCEEM
NDINAPYLVMGQKQGGELVITSVKRWQKGQREFKRISRSIRKLQC.

### Example 6

### Expression of Recombinant Murine and Human SFRP2

For recombinant protein expression in the mammalian expression systems, the cDNA sequence encoding the murine or human SFRP2 is cloned into NheI/MluI or NheI/XbaI sites of a modified pcDNA3.1 vector, so that the expressed protein is tagged with either 6×His or human Fc. After sequence confirmation, the plasmid is tested for expression and secretion by transient transfection of the plasmids into suspension-, serum-free adapted 293T, 293-F, and CHO-S cells using FreeStyle MAX transfection reagent (Invitrogen). The identity of the secreted protein is confirmed by anti-His, Anti-hFc, and/or available gene-specific antibodies. The cell line revealing the highest level of the protein secretion is then selected for large-scale transient production of the protein in spinners and/or Wave Bioreactor® System for 5-7 days. The recombinant protein in the supernatant from the transient production is purified by Ni-NTA beads or Protein A-Sepharose affinity chromatography using ÄKTAexplorer™ (GE Healthcare), and followed by other purification methods, if needed. The purified protein is then dialyzed against PBS, concentrated to ˜1 mg/ml or higher concentrations, and stored at −80° C. until use.

For recombinant protein expression in the bacterial expression system, the cDNA sequence encoding the SFRP2 protein is cloned into NdeI/Hind III or KpnI/Hind III sites of pET30(+) vector, so that the expressed protein is tagged with 6×His. The sequencing confirmed plasmid is transformed into BL21(DE3) cells. The protein expression is induced by adding IPTG in the culture and confirmed with anti-His or gene-specific antibodies. If the expressed protein is in soluble fraction, the protein in soluble fraction will be purified by Ni-NTA affinity chromatography followed by other purification methods if needed. If the expressed protein is in inclusion bodies, the inclusion bodies will be isolated first. The protein in the inclusion bodies is denatured using urea or other denaturing reagents, purified by Ni-NTA beads, refolded, and further purified using other methods if needed. Endotoxin level in the purified protein is then examined, and removed by different methods until the endotoxin level is within the acceptable range. The protein is then dialyzed, concentrated and stored as described above.

### Example 7

### Effect of Murine SFRP2 Neutralizing Antibody on Glucose Metabolism in Mice with Diet-Induced Obesity

The ability of antibody to mouse SFRP2 to affect the level of plasma glucose can be tested as follows. Antibody to murine SFRP2 protein and a control antibody dissolved in PBS is injected into mice on high-fat diet at 30 mg/kg, 10 mg/kg, and 3 mg/kg via IP, SC or IV once a day for two weeks. Body weight, 4-hour fasting blood glucose levels are determined one and two weeks after the initiation of injections. Glucose tolerance test is carried out performed in week 2 and serum insulin is also determined in week 2. Assays are performed as described above in Examples 1-4.

### Example 8

### Efficacy Evaluation of Murine SFRP2 Antibody Using Mouse Diet-Induced Obesity Model

The ability of antibodies against murine SFRP2 to regulate the level of plasma glucose can be tested as follows. Antibody targeting murine SFRP2 protein, or control protein, dissolved in PBS is injected into mice on high-fat diet at 30, 10, and 3 mg/kg via IP, SC or IV once a day for two weeks. Body weight, 4-hour fasting blood glucose levels are determined one and two weeks after the initiation of injections. Glucose tolerance test is carried out performed in week 2 and serum insulin is also determined in week 2. Assays are performed as described above in Examples 1-4.

### Example 9

### Treatment of Mice Having Diet-Induced Obesity with Antibodies Against Human SFRP2

To evaluate the effects of antibodies against human SFRP2 on glucose metabolism, mAb106, a monoclonal antibody against human SFRP2, was generated and administered to mice having diet-induced obesity (DIO). Six-week old male C57B/6 mice were subjected to a 60% kcal fat diet for about 12 weeks before receiving subcutaneous injection of mAb106. Mice body weight, blood glucose and plasma insulin concentrations were determined.

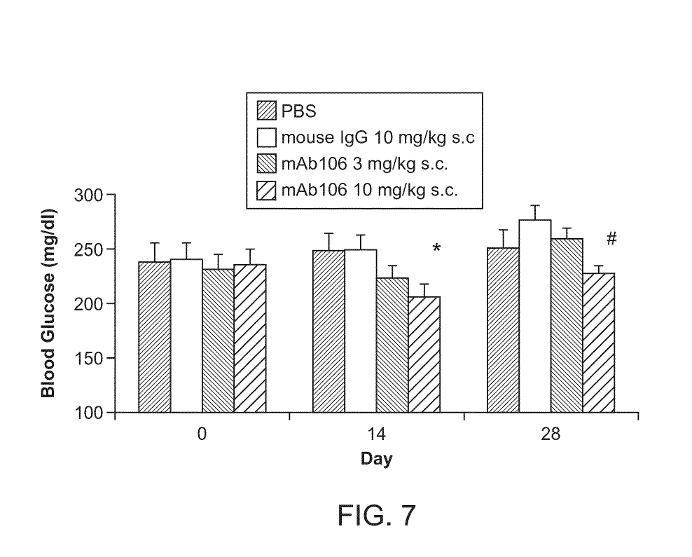

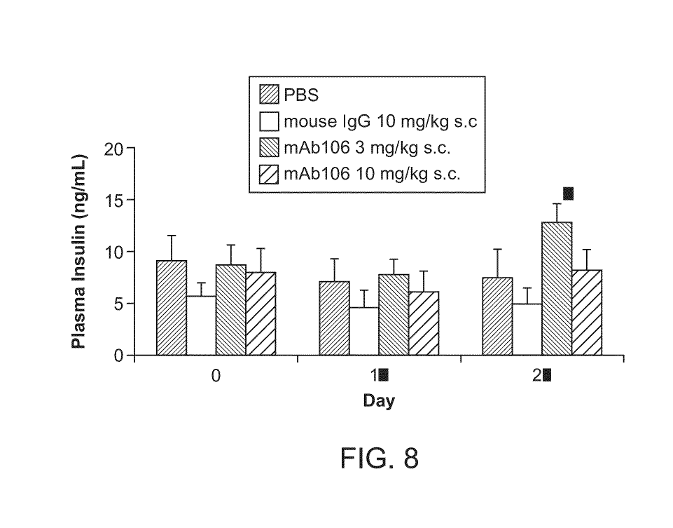

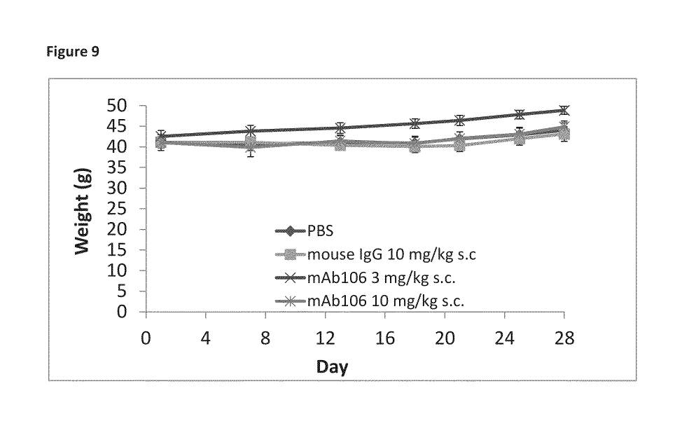

The ability of mAb106 to regulate blood glucose was determined by injecting mAb106 at dosages of 3 mg/kg and 10 mg/kg subcutaneously twice a week (Monday and Thursday) for 4 weeks into mice that had been fed as described above. Blood glucose (FIG. 7) and plasma insulin (FIG. 8) concentrations were measured on days 14 and 28. For FIG. 7: *P<0.05 compared to PBS (t-test); and #p<0.05 compared to mouse IgG (t-test). For FIG. 8: #p<0.05 compared to mouse IgG (t-test). Body weight (FIG. 9) was measured once a week. Mouse IgG was used as a non-specific antibody control for the experiment.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a subject having a glucose metabolism disorder, the method comprising administering to said subject a therapeutically effective amount of an antibody that specifically binds to a protein comprising an amino acid sequence having at least 79% amino acid sequence identity to an amino acid sequence of human SFRP2.

2. The method of claim 1, wherein the treating reduces plasma glucose in said subject.

3. The method of claim 1, wherein the treating reduces plasma insulin in said subject.

4. The method of claim 1, wherein the treating increases glucose tolerance in said subject.

5. The method of claim 1, wherein the glucose metabolism disorder comprises diabetes mellitus.

6. The method of claim 1, wherein the subject is obese.

7. (canceled)

8. The method of claim 1, wherein the subject is a human.

9. The method of claim 1, wherein the administering is by parenteral injection.

10. The method of claim 9, wherein the parenteral injection is subcutaneous.

11. The method of claim 1, wherein the protein comprises an amino acid sequence having at least 85% amino acid sequence identity to an amino acid sequence of human SFRP2.

12. The method of claim 1, wherein the protein comprises an amino acid sequence having at least 90% amino acid sequence identity to an amino acid sequence of human SFRP2.

13. The method of claim 1, wherein the protein comprises an amino acid sequence having at least 95% amino acid sequence identity to an amino acid sequence of human SFRP2.

14. (canceled)

15. The method of claim 1, wherein the protein comprises an amino acid sequence having at least 98% amino acid sequence identity to an amino acid sequence of human SFRP2.

16. The method of claim 1, wherein the human SFRP2 comprises the human SFRP2 amino acid sequence depicted in FIG. 6.

17. A monoclonal antibody that binds specifically to an SFRP2 polypeptide comprising an amino acid sequence having at least 79% amino acid sequence identity to an amino acid sequence of human SFRP2.

18. The antibody of claim 17, wherein the antibody comprises a light chain variable region and a heavy chain variable region present in separate polypeptides.

19. The antibody of claim 17, wherein the antibody comprises a light chain variable region and a heavy chain variable region present in a single polypeptide.

20. The antibody of claim 17, wherein the antibody binds the SFRP2 polypeptide with an affinity of from about 10⁷ M⁻¹ to about 10¹² M⁻¹.

21. The antibody of claim 17, wherein the antibody comprises a heavy chain constant region, and wherein the heavy chain constant region is of the isotype IgG1, IgG2, IgG3, or IgG4.

22. The antibody of claim 17, wherein the antibody is detectably labeled.

23. The antibody of claim 17, wherein the antibody is a Fv, scFv, Fab, F(ab′)2, or Fab′.

24. The antibody of claim 17, wherein the antibody comprises a covalently linked non-peptide polymer.

25. The antibody of claim 24, wherein the covalently linked non-peptide polymer is poly(ethyleneglycol) polymer.

26. The antibody of claim 17, wherein the antibody comprises a covalently linked moiety selected from a lipid moiety, a fatty acid moiety, a polysaccharide moiety, and a carbohydrate moiety.

27. The antibody of claim 17, wherein the antibody comprises an affinity domain.

28. The antibody of claim 17, wherein the antibody is immobilized on a solid support.

29. The antibody of claim 17, wherein the antibody is a humanized antibody.

30. The antibody of claim 17, wherein the antibody is a single chain Fv (scFv) antibody.

31. The antibody of claim 30, wherein the scFv is multimerized.

32. The antibody of claim 17, wherein the SFRP2 polypeptide comprises an amino acid sequence having at least 85% amino acid sequence identity to an amino acid sequence of human SFRP2.

33. The antibody of claim 17, wherein the SFRP2 polypeptide comprises an amino acid sequence having at least 90% amino acid sequence identity to an amino acid sequence of human SFRP2.

34. The antibody of claim 17, wherein the SFRP2 polypeptide comprises an amino acid sequence having at least 95% amino acid sequence identity to an amino acid sequence of human SFRP2.

35. (canceled)

36. The antibody of claim 17, wherein the SFRP2 polypeptide comprises an amino acid sequence having at least 98% amino acid sequence identity to an amino acid sequence of human SFRP2.

37. The antibody of claim 17, wherein the human SFRP2 comprises the human SFRP2 amino acid sequence depicted in FIG. 6.

38. A pharmaceutical composition comprising.
a) the antibody of claim 17; and
b) a pharmaceutically acceptable excipient.

39. (canceled)

40. The composition of claim 38, wherein the composition is suitable for human administration.

41. A sterile container comprising the composition of claim 38.

42. The container of claim 41, wherein the container is a syringe.

43. A kit comprising the sterile container of claim 41.

44. The kit of claim 43, further comprising a second sterile container comprising a second therapeutic agent.
